(19) United States
(12) Patent Application Publication
(10) Pub. No.: US 2023/0107937 A1
(43) Pub. Date: Apr. 6, 2023

(54) ZWITTERIONIC POLYPEPTIDE AND DERIVATIVE THEREOF AND NANODRUG BASED THEREON

(71) Applicant: ZHEJIANG UNIVERSITY, Hangzhou (CN)

(72) Inventors: Shengfu CHEN, Hangzhou (CN); Weili XUE, Hangzhou (CN); Ashish TRITAL, Itahari (NP)

(21) Appl. No.: 17/986,922

(22) Filed: Nov. 15, 2022

Related U.S. Application Data
(63) Continuation of application No. PCT/CN2021/074131, filed on Jan. 28, 2021.

(30) Foreign Application Priority Data
May 17, 2020 (CN) 202010416420.3

(51) Int. Cl. A61K 47/64; A61P 35/00; A61K 9/107

(52) U.S. Cl. CPC A61K 47/645; A61P 35/00; A61K 9/1075

(57) ABSTRACT

The present application discloses a zwitterionic polypeptide and a derivative thereof and a nanodrug based thereon. The nanodrugs can be prepared based on the zwitterionic polypeptide or derivatives thereof. The secondary structure of the zwitterionic polypeptide in the nanodrugs has excellent conversion ability before and after drug release, which can accelerate the release of drugs in cells. The prepared nanodrug can be used in tumor targeted therapy to achieve unexpected tumor targeting with excellent capability in blood compatibility, immune recognition escaping, tumor cell internalization and nucleus targeting, and thus reduces the biodistribution of the nanodrug in liver, kidney, spleen, lung, heart and other health organs which have plenty of reticuloendothelial tissues. Consequently, the prepared nanodrug can effectively inhibit tumor growth with low toxicity in vivo.

### CROSS-REFERENCE TO RELATED APPLICATIONS

The present application is a National Stage of International Application No. PCT/CN2021/074131, filed Jan. 28, 2021, which claims priority to Chinese Patent Application No. 202010416420.3, titled with “ZWITTERIONIC POLYPEPTIDE AND DERIVATIVE THEREOF, AND NANO-DRUG BASED ON SAME” and filed on May 17, 2020, the contents of which are incorporated herein by reference in their entireties.

### TECHNICAL FIELD

The application relates to a zwitterionic polypeptide and a derivative thereof and a nanodrug based thereon, belonging to the field of functional polypeptide and nanodrug manufacturing.

### BACKGROUND

Malignant tumor (or cancer), as a chronic disease worldwide, has the characteristics of abnormal proliferation of cells. After half a century of huge investment, cancer is still a great threat to the human health although some progress has been made in the medical treatment of cancer. According to the International Agency for Research on Cancer, it remains a major threat to mankind. And there will be 22.2 million new cases of cancer and 13.2 million deaths by 2030. Chemotherapy is one of the major methods for cancer treatment. However, it has three key unsolved problems, including the side effects of toxification on the cells in healthy organs and normal tissues, the multi-drug resistance after long-term treatment, and the low bioavailability in cancer caused by physical and chemical properties of chemotherapeutic drugs. In particular, the side effects of toxification on the cells in healthy organs and normal tissues further limits the therapeutic dosage of chemotherapy drugs, which makes them more susceptible to cause multidrug resistance, and thus ultimately leads to the failure of tumor treatment. Therefore, it is urgent to develop a highly effective tumor-targeting drug delivery to reduce the biodistribution of chemotherapeutic drugs in healthy organs and tissues, which might revolutionize the cancer treatment.

Although nanodrugs have always been considered as a feasible means for targeting chemotherapy, effective tumor-targeting nanodelivery has not been achieved so far due to the intricacy of multiple biological barriers in vivo. These multiple biological barriers include: 1) escaping the clearance mechanisms to buy necessary time for turmor targeting, which includes the blood compatibility, the phagocytosis by the reticuloendothelial system and kidney filtration, 2) the penetration of vascular barrier, 3) the improvement of targeting ability, 4) the improvement of the penetration in tumor tissues, 5) the enhancement of cytomembrane crossing of tumor cells, 6) the escaping of the degradation caused by intracellular digestion and efflux mechanism, 7) the ability to enter the nucleus or other organelles where drug molecule act as inhibitor. Moreover, only a unified nanodrug, which can overcome all these barriers, will achieve effective treatment of tumor. Therefore, it is very difficult to achieve targeting nanodelivery. However, protein molecules (such as antibodies), which can be regarded as the most effective nanodrugs, have the natural ability to overcome the first four biological barriers mentioned above. When combining with the ability to effectively cross cytomembrane barriers, escape intracellular digestion and efflux, and enter drug-inhibiting organelles, a nanodrug, which also mimics the targeting of protein molecules, will enable effective targeted therapies. In fact, there are numerous studies focusing on penetrating through each barrier mentioned above. However, it is a great challenge to integrate all capabilities in one nanodrug system to overcome the above seven barriers. Thus, up-to-date, there lacks targeted nanodrugs with excellent performance.

To achieve targeting nanodelivery, the materials of nanocarriers must have good resistance to nonspecific protein adsorption, which include two main types of materials, hydrogen-bond-based hydrophilic ones, and zwitterionic ones. Hydrophilic polyethylene glycol (PEG)-protected nanodrugs show obvious advantages in overcoming the first three barriers, while have a serious weakness in cell membrane barrier penetration. Thus, PEG-protected nanodrugs are often designed to be extraordinarily complex ones to enhance internalization through various additional responsive capabilities. However, it becomes a bottleneck to combine these additional functions through controlling the distribution of functional groups in a nano scale.

Therefore, zwitterionic materials has higher blood compatibility and biocompatibility since they have stronger resistance to nonspecific protein adsorption than PEG and also the zwitterionic property of protein molecules and cell membranes. However, zwitterionic materials are often synthetic polymers, such as polymethacrylate zwitterionic polymers, which face the problem in biodegradability and metabolizability. On the other hand, the studies on zwitterionic materials mostly focused on the state of equal positive and negative charges. Thus, there are limitations in understanding the protein molecules with negative charge bias, thereby lacking a reasonable explanation for the retention of albumin in tumors. In short, zwitterionic polypeptide-based nanodrugs, which mimic negative-charge-biased serum protein molecules, have great potential in tumor chemotherapy.

However, albumin, which shows retention in tumors, can still be highly distributed in liver, kidney and other organs due to its small molecular size. On the other hand, the hydrophobic core of albumin interferes with the encapsulation capacity of small molecule drugs, while the biological function of its hydrophobic core is unknown. Thus, albumin-mimiking, zwitterionic polypeptide-based nanodrugs, which are prepared through eliminating redundant hydrophobic structures in albumin and of which particle size is appropriately enlarged, may exceed the efficacy of albumin encapsulated paclitaxel drugs.

### SUMMARY

The technical problem to be solved by the present application is to provide a new zwitterionic polypeptide, a derivative thereof and a nanodrug based thereon.

To solve the above technical problem, the technical solution adopted by the present application is that the prepared zwitterionic polypeptide has the following chemical structure represented by formula (I):

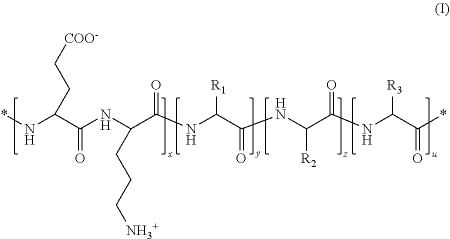

where, x, y, z and u each independently are positive integers, x≥2, y+z+u is greater than 0; R₁ is —CH₂SH and/or a derivative thereof; R₂ is —CH₂COO⁻ and/or an anionic derivative of —CH₂SH; R₃ is a group used to facilitate the degradation of peptides by enzymes.

Furthermore, the zwitterionic polypeptide described in the application has any one of the following structures showed as from formula II to formula VI:

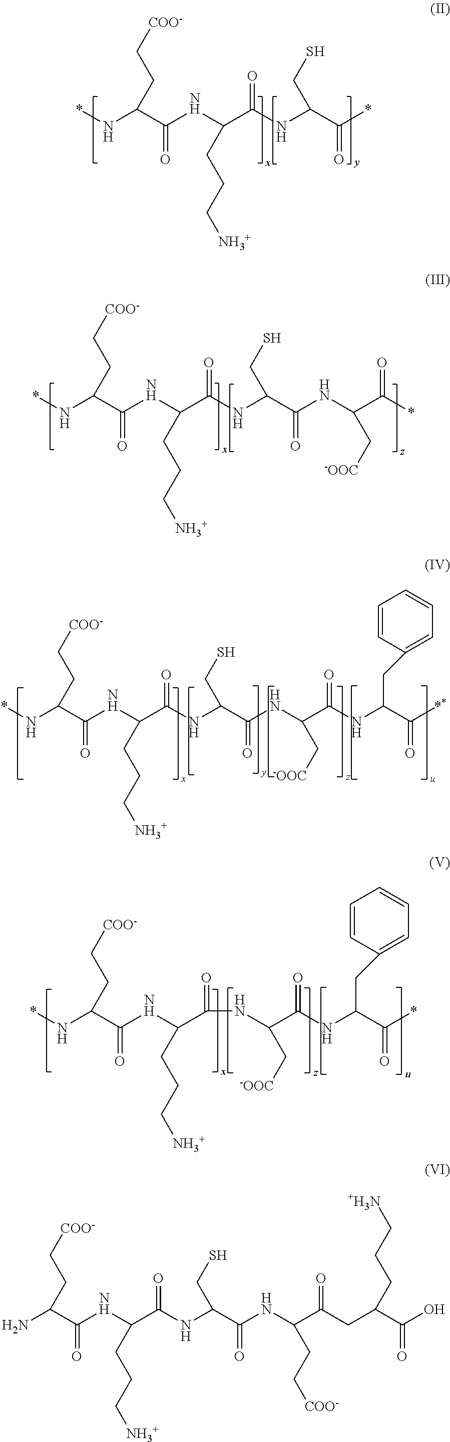

where, x, y, z and u each independently are positive integers, x≥2.

The derivative of the zwitterionic polypeptides described in the application is where,

the derivative of the zwitterionic polypeptide represented by formula II has a structure represented by formula VII or formula VIII;

the derivative of the zwitterionic polypeptide represented by formula III has a structure represented by formula IX or formula X;

the derivative of the zwitterionic polypeptide represented by formula IV has a structure represented by formula XI or formula XII;

the derivative of the zwitterionic polypeptide described as type VI has a structure represented by formula XIII or formula XIV;

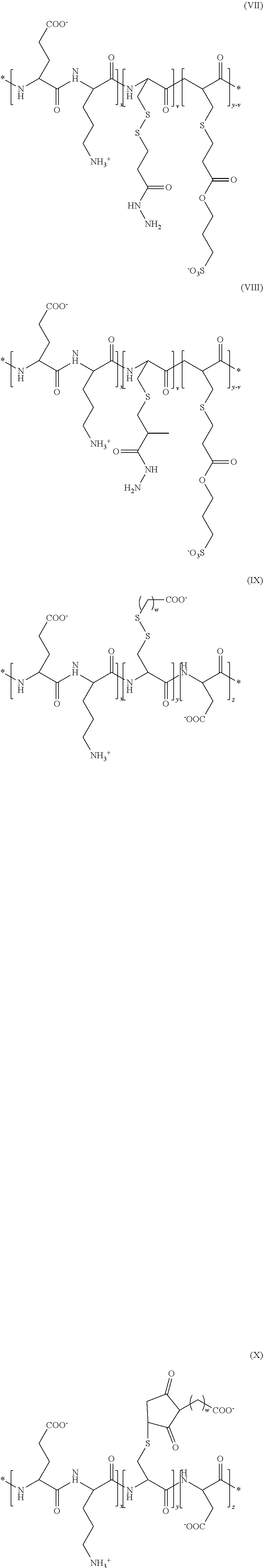

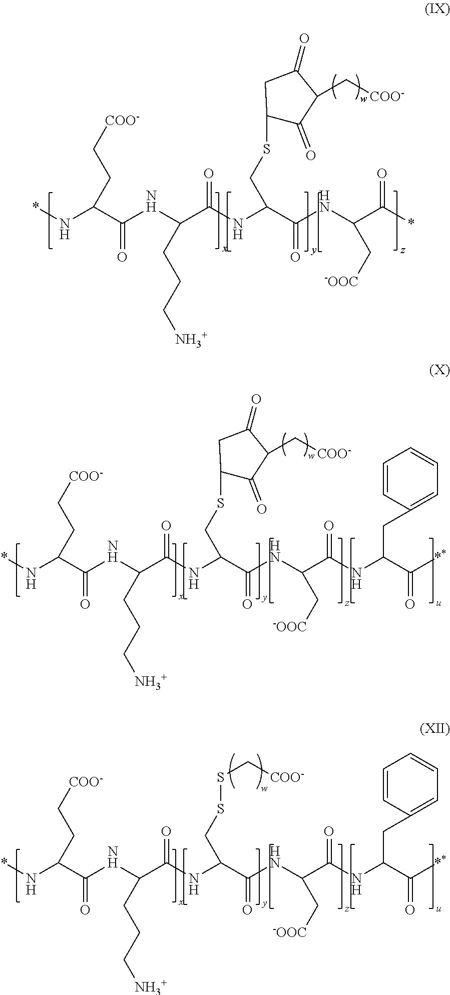

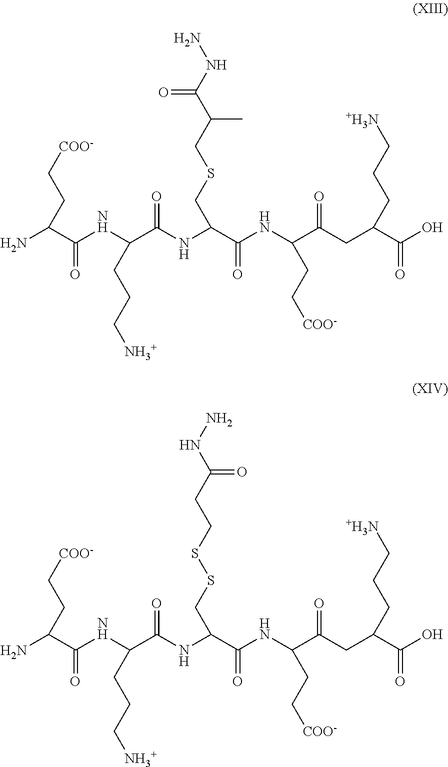

where, x, y, z, u, v and w each independently are positive integers, x≥2, w>6.

Furthermore, a hydrazide group in the derivative represented by formula VII, VIII, XIII or XIV has a structure represented by formula XV after reaction with doxorubicin:

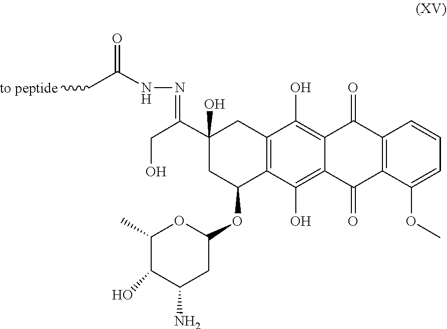

A method of preparing the zwitterionic polypeptides described in the present application includes the following steps of: allowing glutamyl-lysine dipeptide monomer containing side chain protecting groups to undergo mixed polycondensation reaction with any one or several of side-chain protected cysteine, side-chain protected aspartic acid and phenylalanine, and then deprotecting a mixed polycondensation product.

The nanodrug described in the present application is prepared as follows: it is obtained from the encapsulation of the hydrophobic drug by a hydrophobic side chain in the zwitterionic polypeptide represented by formula I and formula IV or formula V, or obtained from the encapsulation of the hydrophobic drug by a hydrophobic side chain in the derivative of the zwitterionic polypeptide represented by formula IX, formula X, formula XI and formula XII.

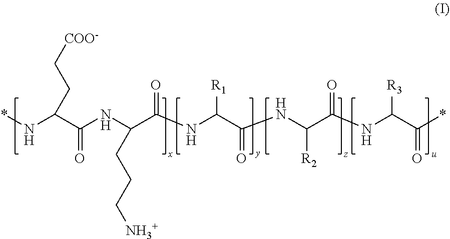

where, x, y, z and u each independently are positive integers, x≥2, y+z+u is greater than 0; R₁ is —CH₂SH and/or a derivative thereof; R₂ is —CH₂COO⁻ and/or an anionic derivative of —CH₂SH; R₃ is a group used to facilitate the degradation of peptides by enzymes;

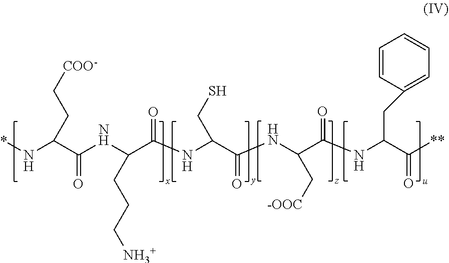

where, x, y, z and u each independently are positive integers, x≥2;

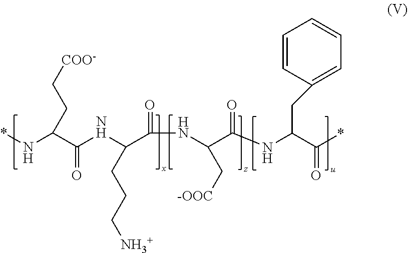

where, x, z and u are positive integers, x≥2;

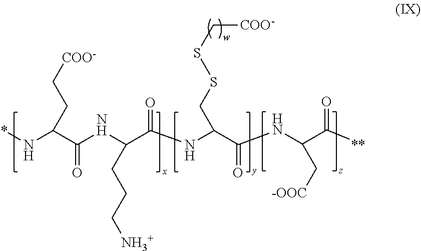

where, x, y, z and w each independently are positive integers, x≥2, w>6;

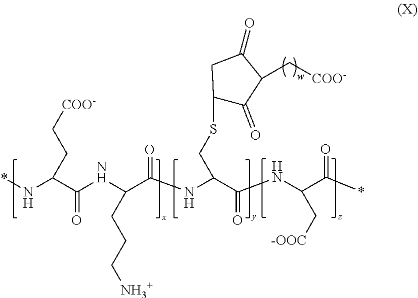

where, x, y, z and w each independently are positive integers, x≥2, w>6;

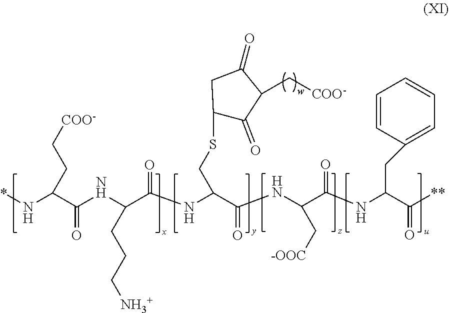

where, x, y, z, u and w each independently are positive integers, x≥2, w>6;

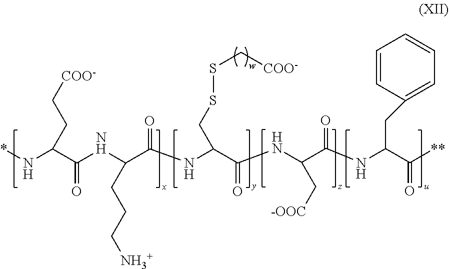

where, x, y, z, u and w each independently are positive integers, x≥2, w>6.

Furthermore, the nanodrug, obtained from the encapsulation of the hydrophobic drug by the hydrophobic side chain in the zwitterionic polypeptide represented by formula I and formula IV or formula IX, or obtained from the encapsulation of the hydrophobic drug by the hydrophobic side chain in the derivative of the zwitterionic polypeptide represented by formula X, formula XI or formula XII, has a zwitterionic polypeptide-protective shell, which is formed by the cross-linking between cysteines in the zwitterionic polypeptides or their derivatives.

The nanodrug is obtained from the encapsulation of the hydrophobic drug by the hydrophobic side chain in the derivatives of the zwitterionic polypeptide represented by formula XV, or formed by directly dispersing the derivative of the zwitterionic polypeptides represented by formula XV in a water solution.

Furthermore, a zeta potential of the nanodrug in a physiological solution of pH 7.4 is negative, and the zeta potential of the nanodrug in a 0.02 mol/L disodium phosphate-citric acid buffer solution of pH 6.7 is below 8 mV.

Furthermore, at least one negatively charged acid group in the nanodrug is more acidic than the carboxylic acid group of glutamate side chain, and/or a total amount of negatively charged acid groups in the nanodrug is more than that of positively charged groups.

The zwitterionic polypeptide described in the present application can be applied in preparing a nanodrug for inhibiting tumor growth.

The derivative of the zwitterionic polypeptide described in the present application can be applied in preparing a nanodrug for inhibiting tumor growth.

The nanodrug based on these zwitterionic polypeptide or derivative thereof described in the present application can be used as a therapeutic drug for inhibiting tumor growth via intravenous injection.

Compared with the prior arts, the advantages of the present application are as follows:

(1) The zwitterionic polypeptide of the application has excellent and stable ability to encapsulate a hydrophobic drug. The drug release amount from the nanodrug based on these zwitterionic polypeptide is less than 7-20% within 24 hours under normal physiological pH7.4 conditions.

(2) The zwitterionic polypeptide has the ability of accelerating drug release after entering tumor tissues and tumor cells due to the change of beta turns with encapsulated drugs to alpha helix during drug releasing, so that the nanodrug can achieve better tumor-cell growth inhibition. This suggested the secondary structure of the zwitterionic polypeptide in the present application has excellent capability of conformational conversion before and after drug release, which can accelerate drug release in cells.

(3) The special zwitterionic glutamine-lysine residue pairs of the zwitterionic-polypeptide-based nanodrug enables the acceleration of the cell internalization and the nucleus entrance of the nanodrug, which makes it easier for the drug to enter the cells.

(4) The zwitterionic-polypeptide-based nanodrug of the application has excellent blood compatibility, and capabilities in escaping immune recognition and reducing the distribution in liver, kidney, spleen, lung, heart and other organs rich in reticuloendothelial tissue through simulating the surfaces of protein molecules in blood, removing the redundant structure in the protein molecules, adding the chemical structure necessary for drug loading, giving the negative charge bias, and enlarging the particle size of the nanodrug over the size of kidney filtration. Moreover, the nanodrug can instantaneously respond to small pH changes (from pH7.4 to pH6.7) to increase the affinity to tumor tissue, which increases the accumulation in tumor tissues and achieves the unexpected capability of tumor targeting. On the other hand, the drug loading content can be elevated, and the carrier is more stable to reduce the premature release before reaching tumor tissues.

(5) The zwitterionic-polypeptide-based nanodrug of the application improves the selective enzymatic degradation of the nanocarrier material at the tumor site by adding enzymatic degradable amino acid residues or sequences, thereby increasing the efficiency of drug delivery to tumor cells. Moreover, the carrier materials are given nature metabolic ability, which reduces the side-effect on the growth of normal tissues. Last, the fully synthetic peptides avoid the application of other biological sources, especially animals, which reduce the possibility of contamination by other pathogens, and make the nanodrug safer.

### BRIEF DESCRIPTION OF DRAWINGS

FIG. 1 shows ¹H NMR spectra of p(EK-co-C) polypeptide with Z protection.

FIG. 2 shows ¹H NMR spectra of negative-charge-biased p(EK-co-C) polypeptide with hydrazide groups via disulfide bonds.

FIG. 3 shows ¹H NMR spectra of negative-charge-biased p(EK-co-C) polypeptide with hydrazide groups via thioether bonds.

FIG. 4 shows a typical CMC result of the doxorubicin-conjugated nanodrug based on negative-charge-biased p(EK-co-C) polypeptide with hydrazide groups via disulfide bonds.

FIG. 5 shows a typical Zeta potential result of the doxorubicin-conjugated nanodrug based on negative-charge-biased p(EK-co-C) polypeptide with hydrazide groups via disulfide bonds.

FIG. 6 shows a typical TEM result of the doxorubicin-conjugated nanodrug based on negative-charge-biased p(EK-co-C) polypeptide with hydrazide groups via disulfide bonds.

FIG. 7 shows typical results of the interaction between serum, fibrinogen and the doxorubicin-conjugated nanodrug based on negative-charge-biased p(EK-co-C) polypeptide with hydrazide groups via disulfide bonds.

FIG. 8 shows typical acidic pH responsive results of the doxorubicin-conjugated nanodrug based on negative-charge-biased p(EK-co-C) polypeptide with hydrazide groups via disulfide bonds.

FIG. 9 shows typical reduction and pH triggered drug releases of the doxorubicin-conjugated nanodrug based on negative-charge-biased p(EK-co-C) polypeptide with hydrazide groups via disulfide bonds.

FIG. 10 shows typical circular dichroism spectra of the doxorubicin-conjugated nanodrug based on negative-charge-biased p(EK-co-C) polypeptide with hydrazide groups via disulfide bonds.

FIG. 11 shows typical cytotoxicity results of the p(EK-co-C) polypeptide with hydrazide groups via disulfide bonds.

FIG. 12 shows typical cytotoxicity results of the doxorubicin-conjugated nanodrug based on negative-charge-biased p(EK-co-C) polypeptide with hydrazide groups via disulfide bonds.

FIG. 13 shows typical MCF-7 cell uptakes of the doxorubicin-conjugated nanodrug based on negative-charge-biased p(EK-co-C) polypeptide with hydrazide groups via disulfide bonds.

FIG. 14 shows typical resistance to immune phagocytosis (RAW-264.7) of the doxorubicin-conjugated nanodrug based on negative-charge-biased p(EK-co-C) polypeptide with hydrazide groups via disulfide bonds.

FIG. 15 shows typical in vivo clearance results of the doxorubicin-conjugated nanodrug based on negative-charge-biased p(EK-co-C) polypeptide with hydrazide groups via disulfide bonds.

FIG. 16 shows typical in vivo biodistribution results of the doxorubicin-conjugated nanodrug based on negative-charge-biased p(EK-co-C) polypeptide with hydrazide groups via disulfide bonds.

FIG. 17 shows typical in vivo antitumor efficacy results of the doxorubicin-conjugated nanodrug based on negative-charge-biased p(EK-co-C) polypeptide with hydrazide groups via disulfide bonds. (a) volume change of tumors bearing on nude mice, (b) body weight change of the mice.

### DESCRIPTION OF EMBODIMENTS

The application will be described below in detail with reference to the attached drawings and specific examples.

The main synthesis method of the zwitterionic polypeptide in the present application is: allowing glutamyl-lysine dipeptide monomer containing side chain protecting groups to undergo mixed polycondensation reaction with any one or several of side-chain protected cysteine, side-chain protected aspartic acid and phenylalanine, and then deprotecting the mixed polycondensation product. Small molecular weight peptides can directly use commercial solid phase synthesis method. The typical synthesis routes are as follows:

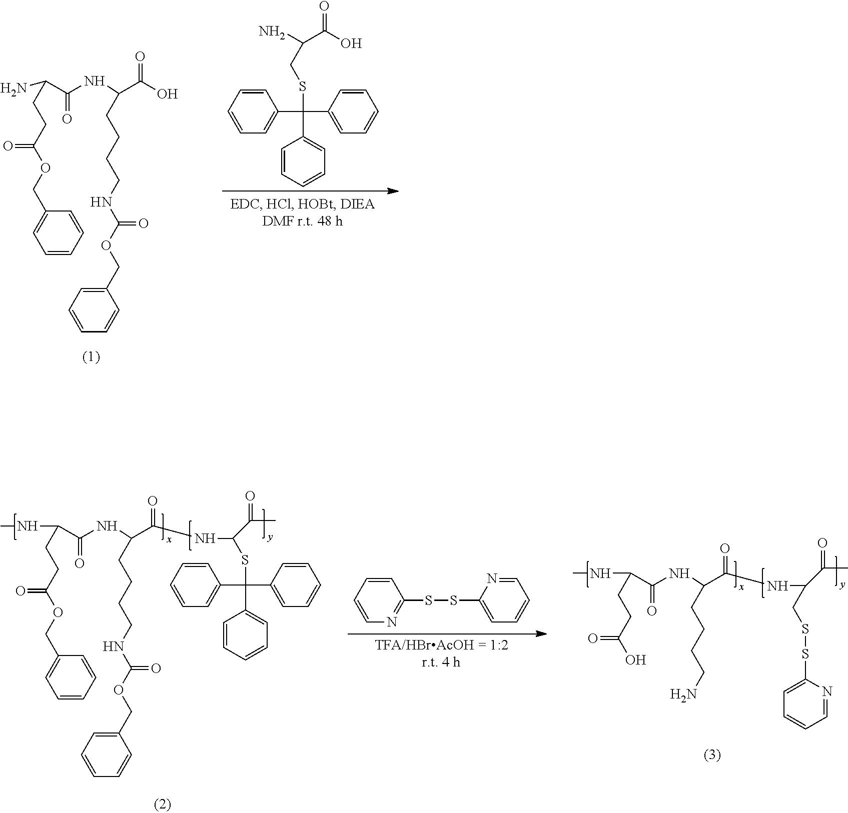

### (1) Synthesis Route of p(EK-C) Polypeptide with Activated Thiol Groups

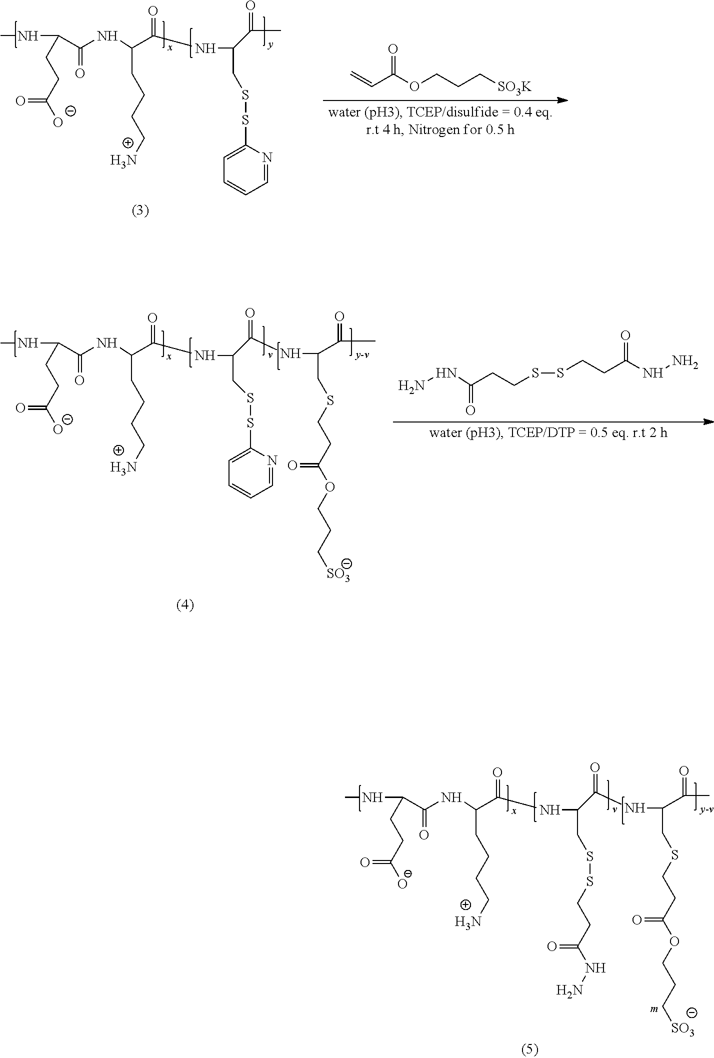

### (2) Synthesis Route of Functional Modification of p(EK-C) for Drug Loading Groups via Disulfide Bonds

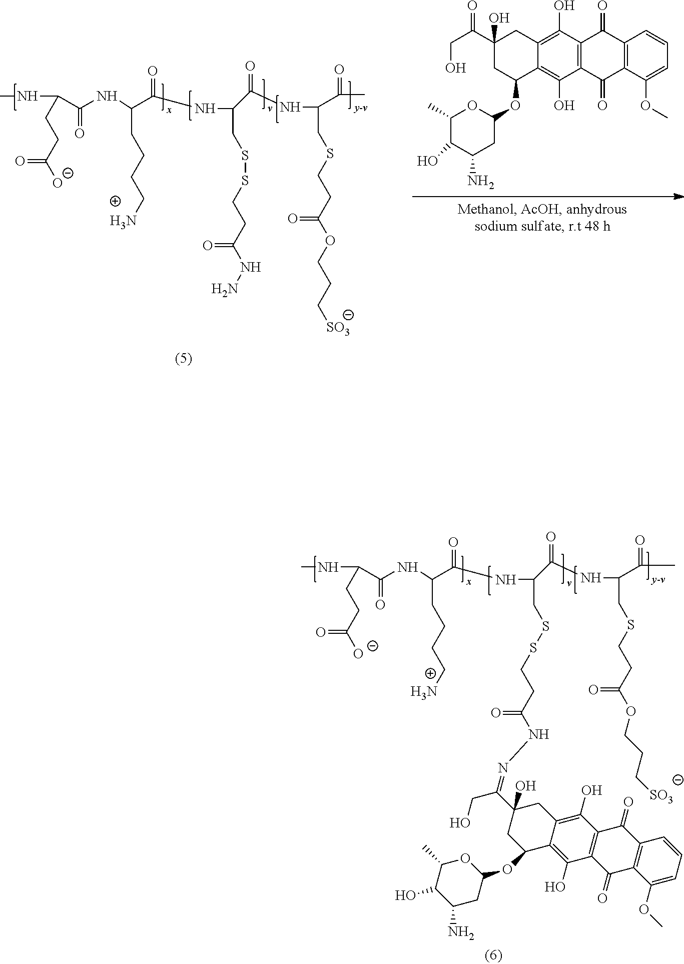

### (3) Synthesis Route of the Conjugation of Antitumor Drug DOX to p(EK-C) via Disulfide Bonds

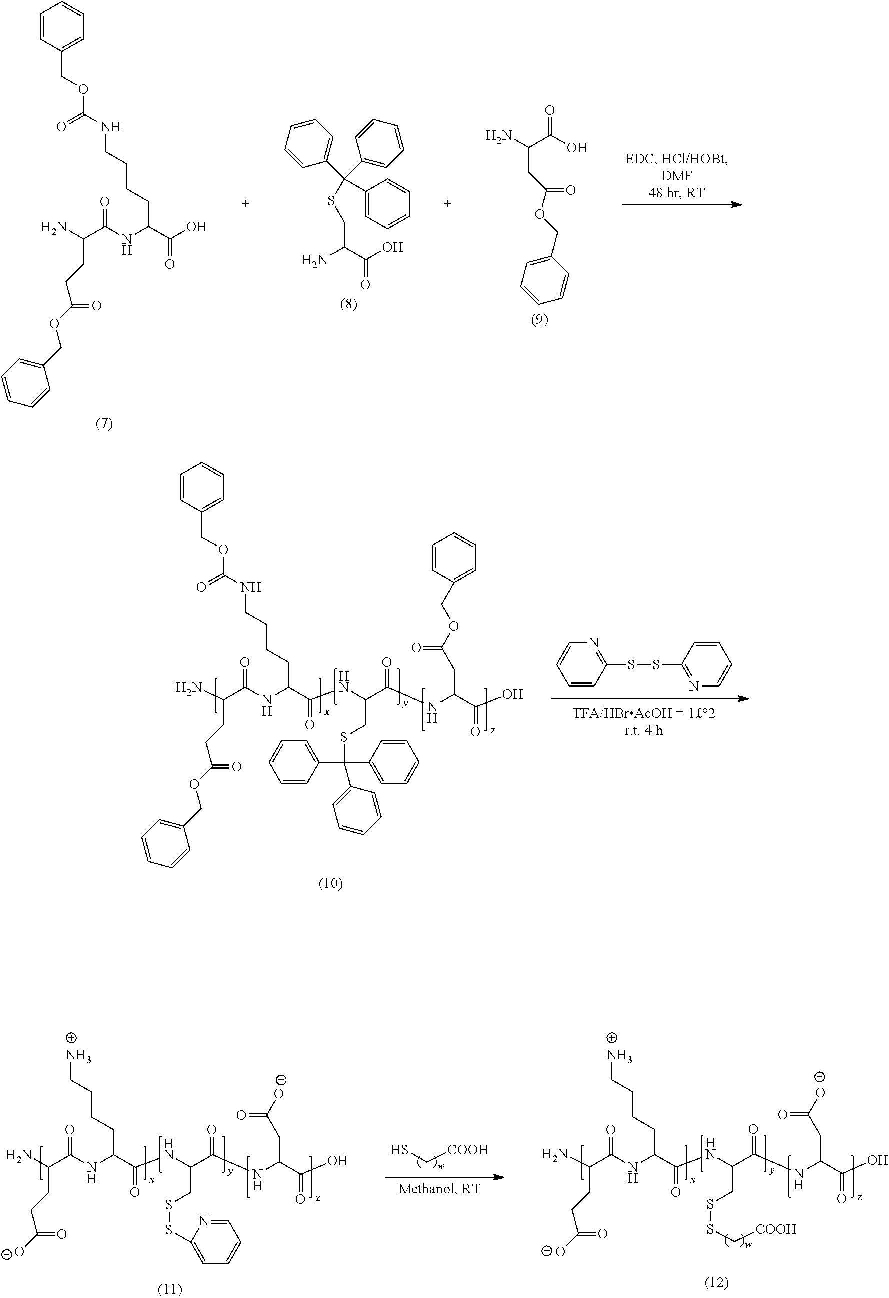

### (4) Synthesis Route of the Alkyl Acid Modified p(EK-C-D-T) from p(EK-C-D) Polypeptide via Disulfide Bonds

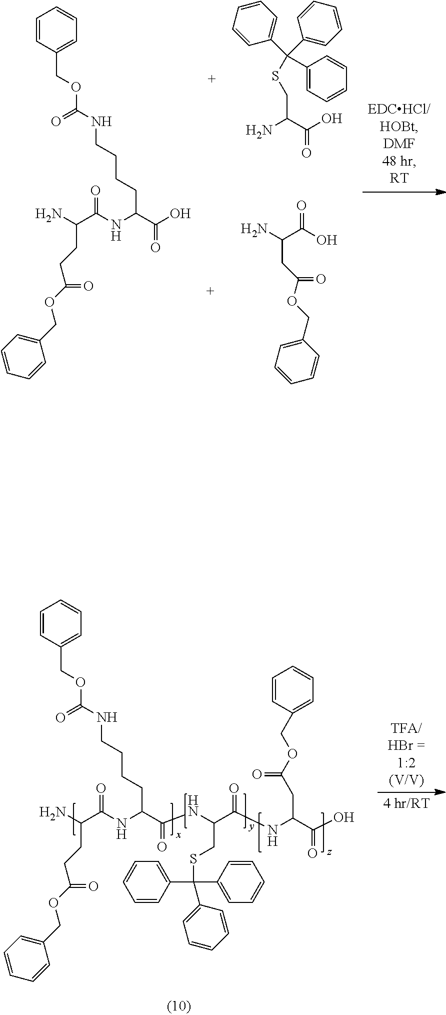

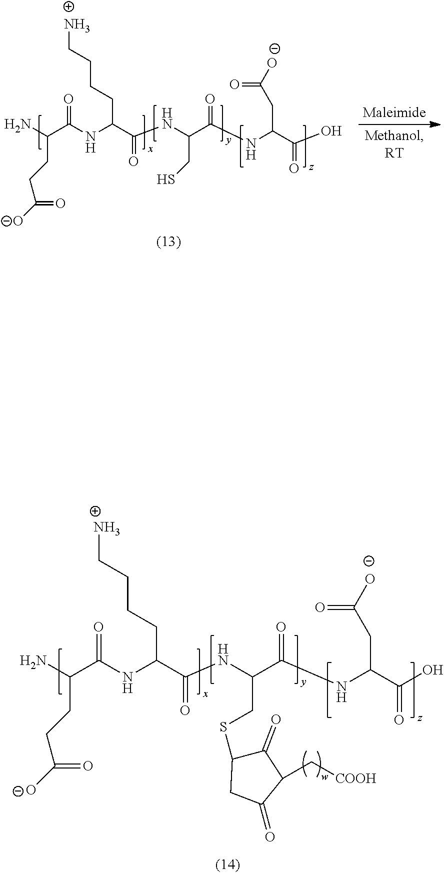

### (5) Synthesis Route of the Alkyl Acid Modified p(EK-C-D-M) from p(EK-C-D) Polypeptide via Thioether Bonds

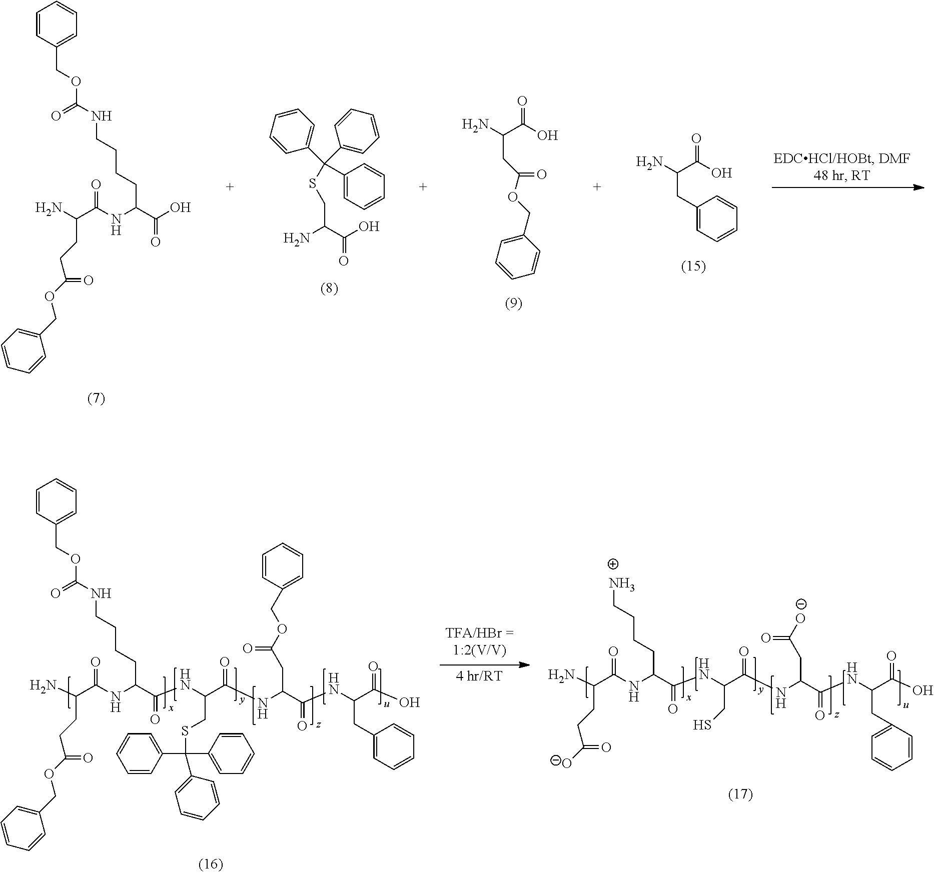

### (6) Synthesis Route of p(EK-C-D-F) Polypeptide

The methods of drug loading by the zwitterionic polypeptides in the present application are mainly implemented in two ways: one is to conjugate drug molecules via environmentally sensitive chemical bonds in cells, for example, acid-sensitive hydrazone bond formed by reaction of hydrazine and ketone group on doxorubicin or reduction-sensitive disulfide bond; the other one is to encapsulate hydrophobic drugs to by hydrophobicity side chain in the zwitterionic polypeptide.

Where, the route of chemical grafting is as follows:

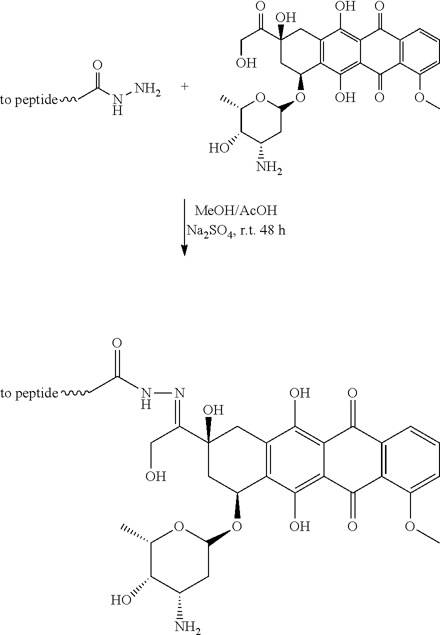

### (7) Synthesis Route of the Conjugation of DOX to the Zwitterionic Polypeptides in the Present Application via Hydrazone Bonds

### EXAMPLE 1

### Preparation of DOX-Loaded Nanodrugs Based on p(EK-C) Polypeptide Synthesized by Glutamyl-Lysine (EK Dimer) and Cysteine (C)

1. Synthesis of the p(EK-C) Polypeptide with Thiopyridine-Activated Thiol Groups.

According to synthesis route (1), 1 g of EK dimer (2 mmol), 0.2422g of H-Cys(Trt)-OH (0.677 mmol), 0.69 g of EDC.HCl (3.6 mmol), 0.4865 g of HOBt (3.6 mmol) and 0.9306 g of DIEA (7.2 mmol) were dissolved in 4 mL of DMF, sealed and stirred for 48 h in dark. The different molar ratios of EK to C in the p(EK-C) polypeptides (EK:C, 2:1, 3:1 and 4:1) were synthesized by changing the feeding ratios of EK dimer to H-Cys(Trt)-OH.

After the reaction was completed, anhydrous ether (10 times of the amount of DMF solution) was added under continuous stirring to remove excess DIEA. The product precipitated after standing a while, and then the supernatant was removed. The methanol solution (10 times of the amount of DMF solution) was added to the flask, and sonicated for 5 min. The sediments were washed by anhydrous methanol two more times to remove the residual salts of HOBt and DIEA. Finally, the sediments were collected after centrifugation and dried to obtain the compound 2, of which the typical NMR spectrum was shown in FIG. 1.

0.2 g of compound 2 and 2,2′-dithiopyridine (PySSPy) (molar ratio of PySSPy to thiol groups in the sidechain of the polypeptide, 3:1) were dissolved in 2 mL of TFA, and then 4 mL of 33 wt. % HBr/HOAc was added and stirred for 4 h. After the reaction was completed, anhydrous ether (10 times of the amount of DMF solution) was added to precipitate the product, and the sediment was washed by anhydrous ether for three times. The sediment was collected after centrifugation and dried for overnight. The crude product was dissolved in water, and ultra-filtrated at molecular weight cut off (MWCO) of 3000 Da to remove the excess PySSPy and other impurities, and lyophilized to obtain compound 3, which was reduced by tris(2-carboxyethyl)phosphine (TECP) to form the zwitterionic polypeptide shown as Formula II (x=32, y=11).

2. Synthesis of p(EK-C)-Based Zwitterionic Polypeptide with Negative Charge Bias and Hydrazide Groups via Disulfide Bond.

According to synthesis route (2), 20 mg of the compound 3 were dissolved in 4 mL of deionized (DI) water. Then, the TCEP with the molar ratio of 1:2 to the sulfydryl sidechain in polypeptide, and potassium 3-sulphonatopropylacrylate (SPP) with the molar ratio of 5:1 to TCEP were added and stirred for 4 hours under N₂ protection. After the reaction was completed, the excess SPP was removed by ultrafiltration.

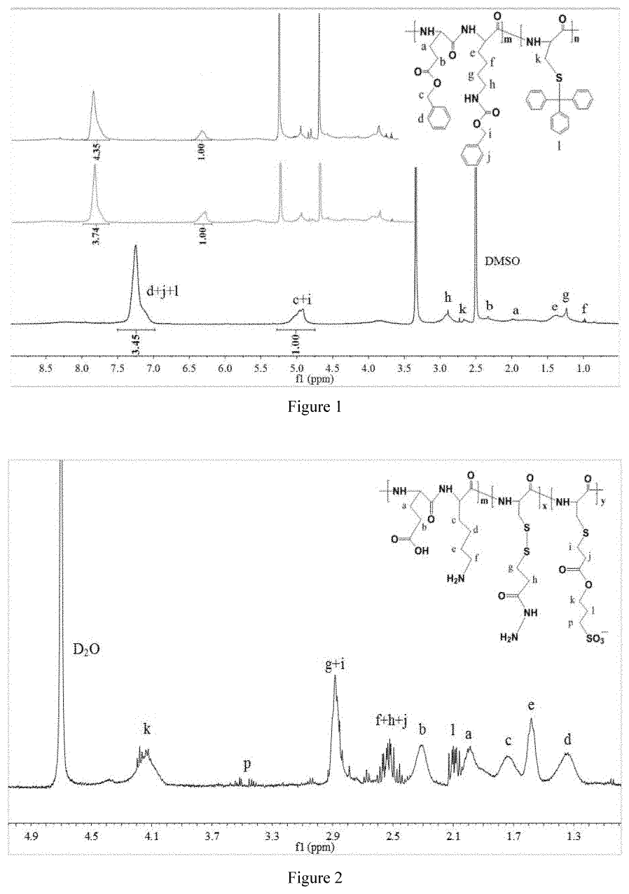

5 mg of 3,3′-dithiobis(propionohydrazide) (DTP) were dissolved in 2 mL of deionized water, and then 3 mg of TCEP were added to reduce the disulfide bonds of DTP with the stirring for 2 hours. After the reaction was completed, excess small molecules such as DTP, TP, TCEP and PySH were removed by ultrafiltration. Finally, the products were lyophilized to obtain the copolymerized polypeptide product (compound 5) with functionalized thiol groups. Namely, the zwitterionic polypeptide derivative represented by formula (VII), where, x=32, v=5, y−v=6, and its typical NMR spectrum was shown in FIG. 2. By adjusting the feeding ratio of DTP and SPP to change their grafting ratios of thiol groups on polypeptide sidechains, the different copolymerized polypeptide products p(EK-C-SS) with the molar ratios of SPP to TP as 2:3, 1:1 and 3:2 were obtained. Namely, the zwitterionic polypeptide derivative shown as formula VII, where, x, v and y−v are set as x=32, v=7, y−v=4; x=32, v=5, y−v=6; x=32, v=4, y−v=7 respectively.

3. Preparations of Conjugates of DOX with Negative-Charge-Biased p(EK-C) Peptide via Disulfide-Bond-Bridged Hydrazide Groups and Nanodrugs Thereof

According to synthesis route (7), 20 mg of the lyophilized powder of the functionalized polypeptide product (compound 5) via the sulfhydryl groups on sidechain were dissolved in 4 mL anhydrous methanol, then 6 mg doxorubicin hydrochloride (DOX.HCl), 4 μL anhydrous acetic acid and appropriate amount of anhydrous sodium sulfate were added. The solution was sealed and stirred for 48 h in dark. After the reaction was completed, the anhydrous sodium sulfate was removed by centrifugation, and the supernatant was ultrafiltered and concentrated at MWCO of 3000 Da to remove the free DOX.HCl. The methanol solution of conjugates (compound 6) of DOX with the polypeptide was concentrated to 0.5 mL. A small amount of TEA was added to the concentrated conjugates solution to remove the HCl on DOX.HCl. Compound 6 has the characteristic structure as represented by formula (XV). The concentrated methanol solution containing compound 6 was added dropwise to 5 mL deionized (DI) water under stirring. The mixture was stirred for another 4 h in dark. Finally, the solution was lyophilized to get the powder of the nanodrug.

Dox in compound 6 can be used as hydrophobic groups. The mix solution of the compound 6 and other hydrophobic drugs is slowly added to pH 8 DI-water, which adjusted by 0.1N NaOH, under stirring. After 4-hour additional stirring, the mixture was dialyzed against borate buffer solution at pH 9.0 for 3 hours and against DI-water for 21 hours and lyophilized to get the nanodrug containing both DOX and other hydrophobic drugs.

4. Synthesis of p(EK-C)-Based Zwitterionic Polypeptide with Negative Charge Bias and Hydrazide Groups via Thioether Bonds

20 mg of the compound 3 were dissolved in 4 mL of DI-water. Then, the TCEP with the molar ratio of 1:2 to the sulfydryl sidechain in polypeptide, and SPP with the molar ratio of 5:1 to TCEP were added and stirred for 4 hours under N₂ protection. After the reaction was completed, the excess SPP was removed by ultrafiltration.

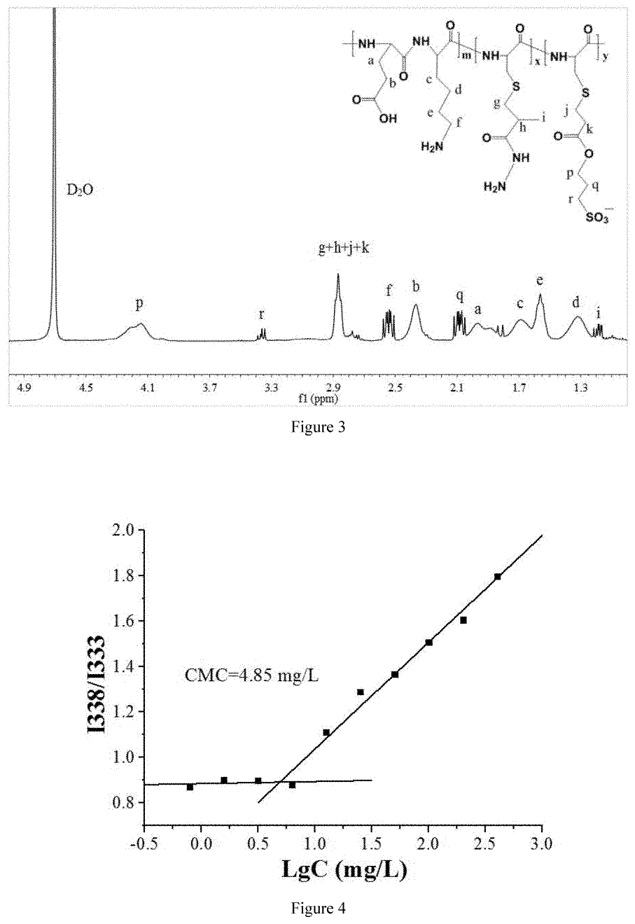

After removing the unreacted SPP by ultrafiltration, additional TCEP was added to reduce the remaining half of the disulfide bond for exposing the sulfhydryl groups on sidechains. Methacryyl hydrazine (MAH) with the molar ratio of 5:1 to the sulfhydryl groups was added and stirred for 4 hours under N₂ protection. After the reaction was completed, the excess MAH was removed by ultrafiltration. Finally, the products were lyophilized to obtain the p(EK-C)-based zwitterionic polypeptides with negative charge bias and hydrazide groups via thioether bond. and the typical NMR spectrum was shown in FIG. 3. By adjusting the feeding ratio of SPP and MAH to change their grafting ratios of thiol groups on polypeptide sidechains, the different copolymerized polypeptide products (p(EK-C-E)) with the molar ratios of SPP to MAH as 2:3, 1:1 and 3:2 were obtained . Namely, the zwitterionic polypeptide derivative shown as formula VIII, where, x,v and y−v are set as x=32, v=7, y−v=4; x=32, v=5, y−v=6; x=32, v=4, y−v=7 respectively.

5. Preparations of Conjugates of DOX with Negative-Charge-Biased p(EK-C) Peptide via Thioether-Bridged Hydrazide Groups and Nanodrugs Thereof

According to synthesis route (7), 20 mg of lyophilized powder of negative-charge-biased p(EK-C) peptide with thioether-bridged hydrazide groups (zwitterionic polypeptide derivatives shown as formula VIII) were dissolved in 4 mL anhydrous methanol, then 6 mg doxorubicin hydrochloride (DOX.HCl) was added, 4 μL anhydrous acetic acid and appropriate amount of anhydrous sodium sulfate were added, and the reaction solution was sealed and stirred for 48 h in dark. After the reaction was completed, the sodium sulfate solid was removed by centrifugation, and the supernatant was ultrafiltered and concentrated at MWCO of 3000 Da to remove the free DOX.HCl. The methanol solution of conjugates of DOX with the polypeptide was concentrated to 0.5 mL. A small amount of TEA was added to the concentrated conjugates solution to remove the HCl on DOX.HCl. The product has the characteristic structure as represented by formula (XV). The concentrated solution containing the products was added dropwise to 5 mL DI-water under stirring. The mixture was stirred for another 4 h in dark. Finally, the solution was lyophilized to get the powder of the nanodrug.

### EXAMPLE 2

### Preparation of DOX-Loaded Nanodrugs Based on p(EK-C-D) Polypeptide Synthesized by Using Glutamyl-Lysine (EK Dimer), Cysteine (C) and Aspartic Acid (D)

1. Synthesis of the p(EK-C-D) Polypeptide with Thiopyridine-Activated Thiol Groups

According to synthesis route (4), 1 g of EK dimer (2 mmol) with side chain protected by Z groups, aspartic acid (0.3 mmol) with side chain protected by Z groups, 0.1817 g of H-Cys(Trt)-OH (0.5 mmol), 0.69 g of EDC.HCl (3.6 mmol), 0.4865 g of HOBt (3.6 mmol) and 0.9306 g of DIEA (7.2 mmol) were dissolved in 4 mL of anhydrous DMF and the solution was sealed and stirred for 48 h in dark. After the reaction was completed, 60 mL of anhydrous ether were added to precipitate the product, and then anhydrous methanol were used to wash the sediment for two times. Finally, the sediment was collected and dried to obtain the compound after centrifugation. Different molar ratios of p(EK-C) polypeptide (EK:C, 2:1, 3:1 and 4:1) were synthesized by changing the feeding ratio of the EK dimer, H-Cys(Trt)-OH and aspartic acid with side chain protected by Z groups.

After the reaction was completed, anhydrous ether (10 times of the amount of DMF solution) was added under continuous stirring to remove excess DIEA. The product precipitated after standing a while, and then the supernatant was removed. The methanol solution (10 times of the amount of DMF solution) was added to the flask, and sonicated for 5 min. The sediments were washed by anhydrous methanol two more times to remove the residual salts of HOBt and DIEA. Finally, the sediments were collected after centrifugation and dried to obtain the compound 10.

0.2 g of compound 10 and 2,2′-dithiopyridine (PySSPy) (molar ratio of PySSPy to thiol groups in the sidechain of the polypeptide, 3:1) were dissolved in 2 mL of TFA, and then 4 mL of 33 wt. % HBr/HOAc was added and stirred for 4 h. After the reaction was completed, anhydrous ether (10 times of the amount of the solution) was added to precipitate the product, and the sediment was washed by anhydrous ether for three times. The sediment was collected after centrifugation and dried for overnight. The crude product was dissolved in water, and ultra-filtrated at MWCO of 3000 Da to remove the excess PySSPy and other impurities, and lyophilized to obtain the p(EK-C-D) polypeptide with thiopyridine-activated thiol groups (compound 11).

2. Preparations of the p(EK-C-D)-Based (p(EK-C-D-T) Peptide with Disulfide-Bond-Bridged Alkyl Acid and Nanodrugs Thereof of DOX Through Encapsulation

According to synthesis route (4), 20 mg of the p(EK-C-D) polypeptide with thiopyridine-activated thiol groups (compound 11) were dissolved in 4 mL of methanol solutions. w-sulfydryl alkyl acid with the molar ratio to activated thiol groups on sidechain of the polypeptide as 1.1:1 was then added and stirred for 4 hours under N₂ protection. After the reaction was completed, excess w-sulfydryl alkyl acid was removed by ultrafiltration. The concentrated solutions were dialyzed for 2 days against DI-water and lyophilized to obtain p(EK-C-D-T) polypeptide (compound 12). The structure of compound 12 was shown as formula IX, where, x=36, y=8, z=6, w=11; ω-sulfydryl alkyl acid represented ω-sulfydryl caproic acid, ω-sulfydryl octanoic acid and ω-sulfydryl lauric acid, respectively.

1 mg of DOX.HCl and TEA (2 times of amount of DOX) were dissolved in DMSO solution and stirred for 6 hours. And then the solution was slowly dropped into a low-speed stirring 5 ml 5.5 mg p(EK-C-D-T) solution in pH 8 DI-water adjusted by 0.1 N NaOH. After stirring for 4 hours, the mixture was dialyzed against borate buffer solution at pH 9.0 for 3 hours and against DI-water for another 21 hours, and then lyophilized to obtain a DOX-loaded p(EK-C-D-T) nanodrug.

3. Preparations of the p(EK-C-D)-Based (p(EK-C-D-M) Peptide with Thioether-Bridged Alkyl Acid and Nanodrugs Thereof DOX Through Encapsulation

According to synthesis route (5), 0.2 g of compound 10 were dissolved in 2 mL of TFA, and then 4 mL of 33 wt. % HBr/HOAc was added and stirred for 4 h. After the reaction was completed, anhydrous ether (10 times of the amount of the solution) was added to precipitate the product, and the sediment was washed by anhydrous ether for three times. The sediment was collected after centrifugation and dried for overnight to obtain the p(EK-C-D) polypeptide (compound 13), of which structure was shown as formula III, where, x=36, y=8 and z=6.

20 mg of p(EK-C-D) polypeptide (compound 13) were dissolved in 4 mL of methanol solutions. ω-maleimide alkyl acid with the molar ratio to thiol groups on sidechain of the polypeptide as 1:1 was then added and stirred for 4 hours under N₂ protection. After the reaction was completed, excess ω-maleimide alkyl acid was removed by ultrafiltration. The concentrated solutions were dialyzed for 2 days against DI-water and lyophilized to obtain p(EK-C-D-M) polypeptide (compound 14). The structure of compound 14 was shown as formula X, where, x=36, y=8, z=6, w=7; ω-maleimide fatty acid represented ω-maleimide octanoic acid and ω-maleimide undecanoic acid.

1 mg of DOX.HCl and TEA (2 times of amount of DOX) were dissolved in DMSO solution and stirred for 6 hours. And then the solution was slowly dropped into a low-speed stirring 5 ml 5.5 mg p(EK-C-D-M) solution in pH 8 DI-water adjusted by 0.1 N NaOH. After stirring for 4 hours, the mixture was dialyzed against borate buffer solution at pH 9.0 for 3 hours and against DI-water for another 21 hours, and then lyophilized to obtain a DOX-loaded p(EK-C-D-M) nanodrug.

### EXAMPLE 3

### Preparation of DOX-Loaded Nanodrugs Based on p(EK-C-D-F) Polypeptide Synthesized by Using Glutamyl-Lysine (EK dimer), Cysteine (C), Aaspartic Acid (D) and Phenylalanine (F)

1. Synthesis of p(EK-C-D-F) Polypeptide

According to synthesis route (6), 1 g of EK dimer (2 mmol) with side chain protected by Z groups, aspartic acid (0.3 mmol) with side chain protected by Z groups, 0.1817 g of H-Cys(Trt)-OH (0.5 mmol), phenylalanine (F) (0.3 mmol), 0.9 g of EDC.HCl (4.7 mmol), 0.64 g of HOBt (4.7 mmol) and 1.21 g of DIEA (9.4 mmol) were dissolved in 4 mL of anhydrous DMF and the solution was sealed and stirred for 48 h in dark. Different molar ratios of EK:C:D:F in p(EK-C-D-F) polypeptide were synthesized by changing the feeding ratios, of which EK:C of 2:1, 4:1 and 6:1 was obtained by adjusting the ratios of EK dimer to H-Cys(Trt)-OH and EK:F of 2:1, 3:1 and 4:1 was obtained by adjusting the ratios of EK dimer to F.

After the reaction was completed, anhydrous ether (10 times of the amount of DMF solution) was added under continuous stirring to remove excess DIEA. The product precipitated after standing a while, then the supernatant was removed. The methanol solution (10 times of the amount of DMF solution) was added to the flask, and sonicated for 5 min. The sediments were washed by anhydrous methanol two more times to remove the residual salts of HOBt and DIEA. Finally, the sediments were collected after centrifugation and dried to obtain the compound 16.

0.2 g of compound 16 were dissolved in 2 mL of TFA, and then 4 mL of 33 wt. % HBr/HOAc was added and stirred for 4 h. After the reaction was completed, anhydrous ether (10 times of the amount of the solution) was added to precipitate the product, and the sediment was washed by anhydrous ether for three times. The sediment was collected after centrifugation and dried for overnight. The crude product was dissolved in water, and ultra-filtrated at MWCO of 3000 Da to remove other impurities, and lyophilized to obtain the p(EK-C-D-F) polypeptide (compound 17). The structure of compound 17 was shown as formula IV, where, x=32, y=7, z=5 and u=5.

2. Preparations of DOX-Loaded Nanodrugs Encapsulated by p(EK-C-D-F) Peptide

1 mg of DOX.HCl and TEA (2 times of amount of DOX) were dissolved in DMSO solution and stirred for 6 hours. And then the solution was slowly dropped into a low-speed stirring 5 ml 5.5 mg p(EK-C-D-F) solution in pH 8 DI-water adjusted by 0.1 N NaOH. After stirring for 4 hours, the mixture was dialyzed against borate buffer solution at pH 9.0 for 3 hours and against DI-water for another 21 hours, and crosslinked cysteine residues in zwitterionic polypeptides by the dissolved oxygen, and then lyophilized to obtain DOX-loaded p(EK-C-D-M) nanodrug.

Similarly, the crosslinking of cysteine residues in zwitterionic polypeptides represented by formula (I) and the residual cysteine residues in zwitterionic polypeptide derivatives represented by formula (IX), formula (X), formula (XI) and formula (XII) can form a stable zwitterionic polypeptide protected layer on nanodrugs.

3. Preparations of the p(EK-C-D-F)-Based p(EK-C-D-F-M) Peptide with Thioether-Bridged Alkyl Acid and Their Nanodrugs of DOX Through Encapsulation

According to synthesis route (6), 20 mg of p(EK-C-D-F) polypeptide (compound 17) was dissolved in 4 mL of methanol solutions. ω-maleimide alkyl acid with the molar ratio to thiol groups on sidechain of the polypeptide as 1:1 was then added and stirred for 4 hours under N₂ protection. After the reaction was completed, excess w-maleimide alkyl acid was removed by ultrafiltration. The concentrated solutions were dialyzed for 2 days against DI-water and lyophilized to obtain p(EK-C-D-F-M) polypeptide. Its structure was shown as formula XI, where, x=32 ,y=7, z=5, u=5, w=7; ω-maleimide fatty acid represented ω-maleimide octanoic acid and ω-maleimide undecanoic acid.

1 mg of DOX.HCl and TEA (2 times of amount of DOX) were dissolved in DMSO solution and stirred for 6 hours. And then the solution was slowly dropped into a low-speed stirring 5 ml 5.5 mg p(EK-C-D-F-M) solution in pH 8 DI-water adjusted by 0.1 N NaOH. After stirring for 4 hours, the mixture was dialyzed against borate buffer solution at pH 9.0 for 3 hours and against DI-water for another 21 hours, and then lyophilized to obtain a DOX-loaded p(EK-C-D-M) nanodrug.

4. Synthesis of the p(EK-C-D-F) Polypeptide with Thiopyridine-Activated Thiol Groups

According to synthesis route (4) and (6), 1 g of EK dimer (2 mmol) with side chain protected by Z groups, aspartic acid (0.3 mmol) with side chain protected by Z groups, 0.3634 g of H-Cys(Trt)-OH (1 mmol), phenylalanine (F) (1 mmol), 0.9 g of EDC.HCl (4.7 mmol), 0.64 g of HOBt (4.7 mmol) and 1.21 g of DIEA (9.4 mmol) were dissolved in 4 mL of anhydrous DMF and the solution was sealed and stirred for 48 h in dark. Different molar ratios of EK:C:D:F in p(EK-C-D-F) polypeptide were synthesized by changing the feeding ratios, of which EK:C of 2:1, 4:1 and 6:1 was obtained by adjusting the ratios of EK dimer to H-Cys(Trt)-OH and EK:F of 2:1, 3:1 and 4:1 was obtained by adjusting the ratios of EK dimer to F.

After the reaction was completed, anhydrous ether (10 times of the amount of DMF solution) was added under continuous stirring to remove excess DIEA. The product precipitated after standing a while, and then the supernatant was removed. The methanol solution (10 times of the amount of DMF solution) was added to the flask, and sonicated for 5 min. The sediments were washed by anhydrous methanol two more times to remove the residual salts of HOBt and DIEA. Finally, the sediments were collected after centrifugation and dried to obtain the product p(EK-C-D-F) with protection groups.

0.2 g of the p(EK-C-D-F) with protection groups and 2,2′-dithiopyridine (PySSPy) (molar ratio of PySSPy to thiol groups in the sidechain of the polypeptide, 3:1) were dissolved in 2 mL of TFA, and then 4 mL of 33 wt. % HBr/HOAc was added and stirred for 4 h. After the reaction was completed, anhydrous ether (10 times of the amount of the solution) was added to precipitate the product, and the sediment was washed by anhydrous ether for three times. The sediment was collected after centrifugation and dried for overnight. The crude product was dissolved in water, and ultra-filtrated at MWCO of 3000 Da to remove the excess PySSPy and other impurities, and lyophilized to obtain the p(EK-C-D-F) polypeptide with thiopyridine-activated thiol groups.

5. Preparations of the p(EK-C-D-F)-Based p(EK-C-D-F-T) Peptide with Disulfide-Bond-Bridged Alkyl Acid and Their Nanodrugs of DOX Through Encapsulation

According to synthesis route (2), 20 mg of the p(EK-C-D-F) polypeptide with thiopyridine-activated thiol groups were dissolved in 4 mL of methanol solutions. ω-sulfydryl alkyl acid with the molar ratio to activated thiol groups on sidechain of the polypeptide as 1:1 was then added and stirred for 4 hours under N₂ protection. After the reaction was completed, excess ω-sulfydryl alkyl acid was removed by ultrafiltration. The concentrated solutions were dialyzed for 2 days against DI-water and lyophilized to obtain p(EK-C-D-F-T) polypeptide. Its structure was shown as formula XII, where, x=32, y=7, z=5, u=5 and w=5. ω-sulfydryl alkyl acid represented ω-sulfydryl caproic acid, ω-sulfydryl octanoic acid and ω-sulfydryl lauric acid, respectively.

1 mg of DOX.HCl and TEA (2 times of amount of DOX) were dissolved in DMSO solution and stirred for 6 hours. And then the solution was slowly dropped into a low-speed stirring 5 ml 5.5 mg p(EK-C-D-T) solution in pH 8 DI-water adjusted by 0.1 N NaOH. After stirring for 4 hours, the mixture was dialyzed against borate buffer solution at pH 9.0 for 3 hours and against DI-water for another 21 hours, and then lyophilized to obtain a DOX-loaded p(EK-C-D-F-T) nanodrug.

### EXAMPLE 4

### Synthesis of p(EK-D-F) and Preparation of DOX-Loaded Nanodrugs Based on p(EK-D-F) Polypeptide

1. Synthesis of p(EK-D-F) Polypeptide

According to synthesis route (6), 1 g of EK dimer (2 mmol) with side chain protected by Z groups, aspartic acid (0.4 mmol) with side chain protected by Z groups, phenylalanine (F) (1 mmol), 0.73 g of EDC.HCl (3.8 mmol), 0.52 g of HOBt (3.8 mmol) and 0.98 g of DIEA (7.6 mmol) were dissolved in 4 mL of anhydrous DMF and the solution was sealed and stirred for 48 h in dark. Different molar ratios of EK:D:F in p(EK-D-F) polypeptide were synthesized by changing the feeding ratios, of which EK:F of 2:1, 3:1 and 4:1 was obtained by adjusting the ratios of EK dimer to F.

After the reaction was completed, anhydrous ether (10 times of the amount of DMF solution) was added under continuous stirring to remove excess DIEA. The product precipitated after standing a while, then the supernatant was removed. The methanol solution (10 times of the amount of DMF solution) was added to the flask, and sonicated for 5 min. The sediments were washed by anhydrous methanol two more times to remove the residual salts of HOBt and DIEA. Finally, the sediments were collected after centrifugation and dried to obtain the p(EK-D-F) polypeptide with protection groups.

0.2 g of the p(EK-D-F) polypeptide with protection groups was dissolved in 2 mL of TFA, and then 4 mL of 33 wt. % HBr/HOAc was added and stirred for 4 h. After the reaction was completed, anhydrous ether (10 times of the amount of the solution) was added to precipitate the product, and the sediment was washed by anhydrous ether for three times. The sediment was collected after centrifugation and dried for overnight. The crude product was dissolved in water, and ultra-filtrated at MWCO of 3000 Da to remove other impurities, and lyophilized to obtain the p(EK-D-F) polypeptide. Its structure was shown as formula V, where, x=37, z=8, and u=19.

2. Preparation of DOX-Loaded Nanodrugs Encapsulated by p(EK-D-F) Polypeptide

1 mg of DOX.HCl and TEA (2 times of amount of DOX) were dissolved in a DMSO solution and stirred for 6 hours. And then the solution was slowly dropped into a low-speed stirring 5 ml 5.5 mg p(EK-D-F) solution in pH 8 DI-water adjusted by 0.1 N NaOH. After stirring for 4 hours, the mixture was dialyzed against a borate buffer solution at pH 9.0 for 4 hours and against DI-water for another 20 hours, and then lyophilized to obtain a DOX-loaded p(EK-D-F) nanodrug.

### EXAMPLE 5

### Preparation of DOX-Loaded Nanodrugs Based on Glutamyl-Lysine Cysteine Glutamyl-Lysine Pentapeptide (EKCEK)

1. Synthesis of EKCEK Pentapeptide with Hydrazide Groups via Disulfide Bonds and Preparations of its Nanodrug of DOX.

EKCEK pentapeptides were prepared through a standard solid-phase synthesis method and their chemical structure was shown as formula VI. 100 mg of EKCEK (0.155 mmol) were dissolved in 5 mL of methanol, and the 2,2′-dithiopyridine (PySSPy) with the molar ratio of 3:1 to the pentapeptides was added and stirred overnight at room temperature. After the reaction was completed, the crude product was precipitated by the anhydrous ether (10 times of amount of the methanol), and then washed by anhydrous ether for three times and dried in vacuum to obtain the thiopyridine-activated EKCEK pentapeptides.

5 mg of DTP were dissolved in 2 mL of methanol, and then 3 mg of TCEP were added to reduce the disulfide bonds of DTP. 12.7 mg of the thiopyridine-activated EKCEK pentapeptides was added for 2-hour stirring. After the reaction was completed, the crude product was precipitated by the anhydrous ether (10 times of amount of the methanol), and then washed by anhydrous ether for three times and dried in vacuum to obtain the EKCEK pentapeptide with a disulfide-bond-bridged hydrazide group. Its structure was shown as formula XIV

According to synthesis route (7), 20 mg of the lyophilized powder of the EKCEK pentapeptide with a disulfide-bond-bridged hydrazide group were dissolved in 4 mL anhydrous methanol, then 16 mg DOX.HCl, 4 μL anhydrous acetic acid and appropriate amount of anhydrous sodium sulfate were added. The solution was sealed and stirred for 48 h in dark. After the reaction was completed, the anhydrous sodium sulfate was removed by filtration. A small amount of TEA was added into the collected filtrate to remove the hydrochloric acid carried by conjugated DOX, resulting hydrophobic side groups. Then the above methanol solution was added dropwise into the aqueous solution at pH 9 under stirring. The mixture was stirred for another 4 h in dark for the self assembly of DOX-conjugated molecules. The above solution was dialyzed in dark to remove free DOX molecules. Finally, the solution was lyophilized to get the powder of the nanodrug. The lyophilized powder will be used in various chemical characterization and in vitro cell experiments in the future.

2. Synthesis of EKCEK Pentapeptide with Hydrazide Groups via Thioether Bond and Preparations of its Nanodrug of DOX.

200 mg of EKCEK (0.31 mmol) powder were dissolved in an aqueous solution at pH 3, and 67 mg of MAH.HBr (0.37 mmol) without BOC protection were added to react for 4 hour under stirring at room temperature. After the reaction was completed, the solution was lyophilized to obtain the product. Its structure was shown as formula (XIII). 50 mg of lyophilized powder were dissolved in anhydrous methanol, then 42.8 mg DOX.HCl, a drop of anhydrous acetic acid and appropriate amount of anhydrous sodium sulfate were added. The solution was sealed and stirred for 48 h in dark. After the reaction was completed, the anhydrous sodium sulfate was removed by filtration. A small amount of TEA was added into the collected filtrate to remove the hydrochloric acid carried by conjugated DOX, resulting hydrophobic side groups. Then the above methanol solution was added dropwise into the aqueous solution at pH 9 under stirring. The mixture was stirred for another 4 h in dark for the self assembly of DOX-conjugated molecules. The above solution was dialyzed in dark to remove free DOX molecules. Finally, the solution was lyophilized to obtain the powder of the nanodrug. The lyophilized powder will be used in various chemical characterizations and in vitro cell experiments in the future.

### EXAMPLE 6

In this example, the nanodrug of the conjugates of DOX with negative-charge-biased p(EK-C) peptide via disulfide-bond-bridged hydrazide groups in example 1 was used to characterize and measure the tumor inhibition efficacy. The nanodrug was synthesized by EK dimer and H-Cys(Trt)-OH at the ratio of EK to C at 3:1, and the ratio of SPP to MAH at 3:2. In the obtained zwitterionic polypeptide derivatives, EK/MAH/SPP was x=32, v=4, y−v=7, and the efficiency of DOX conjugation via hydrazide groups was more than 90%.

1. Measurement of Critical Micelle Concentration (CMC) of the Drug-Loaded Micelles

In this example, the critical micelle concentration (CMC) of drug loaded micelles of the copolymerized polypeptide was measured by using fluorescence probe pyrene. First, the acetone solution of pyrene with a concentration of 1.6×10⁻⁶ M was prepared, and then the prepared acetone solution of pyrene was added to the graduated test tube to volatilize the acetone. The aqueous solution of the derivatives of the zwitterionic polypeptide from 0.4 mg/mL to 7.8×10⁻⁴ mg/mL were prepared. Then the solutions at different concentrations were respectively added into the graduated test tubes containing pyrene with the final concentration of pyrene at 6×10⁻⁷ M, which was incubated at room temperature in dark for 24 hours. Finally, the spectra 300-360 nm of the emitted light was recorded by a fluorescence spectrophotometer. And then the ratio of the fluorescence intensity at 338 nm and 333 nm wavelengths (I₃₃₈/I₃₃₃) was used to determine the CMCs of the derivatives of the zwitterionic polypeptide in water.

The CMC of the DOX-conjugated nanodrugs based on negative-charge-biased p(EK-co-C) polypeptide with hydrazide groups via disulfide bonds was shown in FIG. 4, which was at the level of 4.85 mg/L.

2. Characterization of the Particle Sizes and Zeta Potentials of the Drug-Loaded Micelles

The particle sizes and zeta potentials of the prepared drug-loaded micelles were characterized by Zetasizer Nano-ZS, and the detailed procedure was as follows:

1 mg of lyophilized powder of the drug-loaded nano micelles was dissolved in 5 mL of PBS solution, and 100 μL of the micelle solution was added to the disposable plastic cuvette for determining the particle sizer. Disodium hydrogen phosphate-citric acid buffer solutions (mcllvaine buffer (MB)) at pH of 5.0, 5.6, 6.2, 6.8, 7.4 and 8.0 were prepared, respectively. Then the lyophilized powder of the drug-loaded nano micelles was dissolved by the MB solution to 0.2 mg/ml at corresponding pH. The zeta potentials of the drug-loaded micelles at various pH values were recorded. Instrument parameters: temperature 25° C., the incident angle for light scattering detection 173°.

The typical zeta potential results of the DOX-conjugated nanodrugs based on negative-charge-biased p(EK-co-C) polypeptide with hydrazide groups via disulfide bonds were shown in FIG. 5, which shows that the nanodrug is slightly negatively charged at pH 7.4, and close to neutral at the tumor microenvironment pH 6.7. This transition of zeta potential makes the nanodrug keep good blood compatibility in circulation and reduce the charge repulsion with tumor cells after entering the tumor microenvironment.

3. Morphological Characterization of the Drug-Loaded Micelles

The morphologies of the drug-loaded micelles were characterized by transmission electron microscopy (TEM). The sample preparation method was as follows: an aqueous solution of drug-loaded micelles at 0.2 mg/mL was prepared, and two or three drops of the solution were pipetted on the copper-mesh-supported carbon film. The sample was placed on the filter paper for drying at room temperature. The morphologies of the micelles were investigated by an electron microscope.

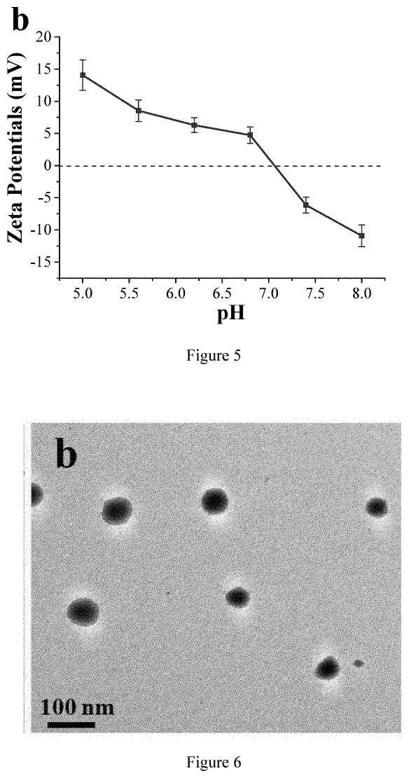

The typical TEM results of the DOX-conjugated nanodrugs based on negative-charge-biased p(EK-co-C) polypeptide with hydrazide groups via disulfide bonds were shown in FIG. 6, showing that the particle size of nanodrug was less than 100 nm.

4. Measurement of the DOX Conjugating Efficiency via the Hydrazide and Drug-Loading Content of the Micelles

Firstly, the standard curve of the 2-mercaptopyridine (PySH) concentration vs UV absorption was established. 2 mg of PySH was dissolved in 3 mL of deionized water (pH 3), then the solution was diluted to the corresponding 5-15 times, and then diluted to 20 times. Finally, the UV absorptions of the diluted solutions were recorded by UV spectrophotometer at 270 nm to obtain the standard curve of PySH in DI-water (pH 3).

Excessive TCEP were added to the aqueous solution of compound 11 to reduce the disulfide bond on the polymer for PySH release, then the absorbance value of the solution was recorded at 270 nm, and the amount of PyS-groups on the polymer (b) was calculated according to the UV absorption standard curve of PySH; next, excess TP were added to the aqueous solution of compound 11 to replace the PyS-groups on the polymer, and then the UV absorbance value of the solution was measured to get the amount of replaced PySH (a). The grafting rate of hydrazide (CE) was calculated according to the following formula: CE=a/b*100%.

Establishment of UV absorption standard curve of DOX: DOX.HCl aqueous solutions with different concentrations were prepared, and then they were acidified by adding a drop of concentrated hydrochloric acid to reduce the influence on DOX.HCl UV absorption, which could be caused by different pH values of the solutions. Finally, the absorbance of the above solutions at 485 nm was measured by UV spectrophotometer to obtain a UV absorption standard curve of DOX.HCl.

1 mg of lyophilized powder of the drug-loaded nano micelles was dissolved in 1 mL of acetic acid buffer solution with pH of 3.0, and then the solution was placed in a metal heater and shaked for 24 hours at 50° C. After the reaction, 2 ml of DI-water acidified with concentrated hydrochloric acid was mixed with the solution in a quartz cuvette, and the absorbance of the sample at 485 nm was recorded. The drug loading capacity (DLC) of drug-loaded micelles was calculated according to the following formula:

DLC=(mass of the DOX in micelles/mass of the micelles)*100%

The typical conjugating rates of the DOX-conjugated nanodrugs based on negative-charge-biased p(EK-co-C) polypeptide with hydrazide groups via disulfide bonds were 95.1%, 90.06% and 91.46% when the ratio of EK to C was 2:1, 3:1 and 4:1, respectively. The conjugating rates of hydrazide group of these three polymers were more than 90%.

5. Stability Measurement of the Drug-Loaded Micelles

Dynamic light scattering (DLS) was used to characterize the stability of drug-loaded micelles by detecting the particle size change with time in different solutions. The detail procedures were as follows: 2 mg of lyophilized powder of the drug-loaded nano micelles was dissolve in 1 mL of PBS solution, then 0.1 mL of this drug-loaded micelle solution was respectively added to 0.9 ml of a PBS solution, a 100% FBS solution and a PBS solution containing 3 mg/ml bovine fibrinogen (Fg). The samples were sealed and stored in a shaking table at 37° C. The samples taken at various time points were used to determine the particle size changes of the drug-loaded micelles.

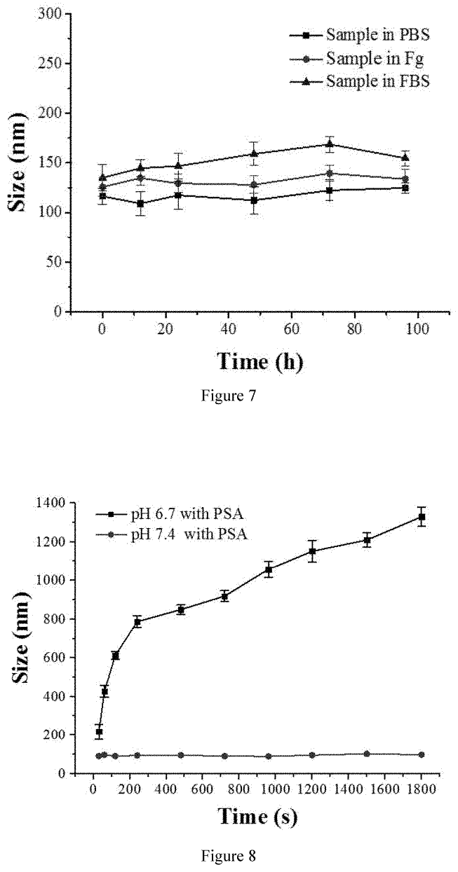

The typical results of the interaction between serum, fibrinogen and the doxorubicin-conjugated nanodrug based on negative-charge-biased p(EK-co-C) polypeptide with hydrazide groups via disulfide bonds were shown in FIG. 7, which shows that the nanodrug is stably compatible with serum and fibrinogen, and will not form aggregation through non-specific protein adsorption.

6. pH Sensitivity Measurement of the Drug-Loaded Micelles

In weak acid environment, the -coo⁻ groups on the surface of drug-loaded micelles will be protonated, thus of enhancing the affinity between the drug-loaded micelles and polysialic acid (PSA). In order to prove the surface-protonation-induced aggregation between the drug-loaded micelles and the overexpressed PSA on tumor cells in TME, 20 mM PB+130 mM NaCl buffer solution (containing 0.075 mg/ml PSA) at pH 6.7 or 7.4 was prepared respectively. The drug-loaded micelle lyophilized powder was dissolved in the above solution with the micelle concentration at 0.2 mg/mL. Finally, the particle size in the mixed solution was recorded by DLS at each time point.

The typical acidic pH responsive results of the doxorubicin-conjugated nanodrug based on negative-charge-biased p(EK-co-C) polypeptide with hydrazide groups via disulfide bonds are shown in FIG. 8, which shows that when the pH is close to the one of TME, PSA on the surface of tumor cells can be recognized to improve the tumor targeting.

7. pH-Triggered and Reductive-Environment-Induced Responsive Drug Release of the Drug-Loaded Micelles

The drug release behavior of the drug-loaded micelles in pH7.4, pH5.0 and pH5.0+GSH (10 mM) buffer solutions was characterized to show the response of drug loaded micelles to the stimulations of TME pH and reductive environment. The detail procedures are as follows: 5 ml of drug loaded micelle solution (5 mg/mL) was added into a dialysis bag at MWCO of 3500 Da, and then respectively placed in 45 mL of the different buffer solutions mentioned above. They were constantly shaken (200 rpm) at 37° C. 2 mL of dialysate was taken out at different time points, and was acidified by concentrated hydrochloric acid. 2 mL of corresponding fresh buffer was added. The absorbance of the dialysate was measured at 485 nm, and the cumulative drug release of drug loaded micelles was calculated by using the DOX standard curve established previously.

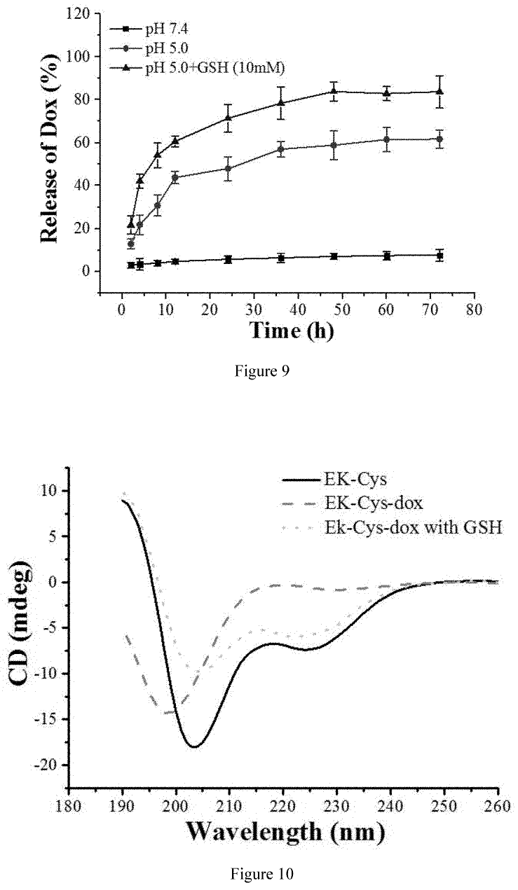

The typical reduction and pH triggered drug releases of the doxorubicin-conjugated nanodrug based on negative-charge-biased p(EK-co-C) polypeptide with hydrazide groups via disulfide bonds are shown in FIG. 9, which shows that the nanodrug is very stable under normal physiological conditions, and has the ability of rapid release in low pH tumor microenvironment and intracellular reduction environment.

8. Circular Dichroism (CD) Spectrum Characterization of the Polypeptide Polymers

The aqueous solution of the polypeptide polymer, the drug-loaded micelle and the drug-loaded micelle+GSH (10 mM) at 0.2 mg/mL was prepared, respectively. 200 μL of the prepared solutions above in a circular dichroic quartz cuvette with an optical path of 1 mm was measured by a circular dichroic spectrometer. The scanning range is 190-260 nm, the scanning speed is 20 nm/min, and the bandwidth is 1.00 nm. The spectra were analyzed by the Dichroweb.

The typical circular dichroism spectra of the doxorubicin-conjugated nanodrug based on negative-charge-biased p(EK-co-C) polypeptide with hydrazide groups via disulfide bonds are shown in FIG. 10, which shows that the conformations of the polypeptides in nanodrugs are mainly random coils and they convert to more loosely alpha helixes after drug release. This indicates that the secondary structure of the zwitterionic polypeptide in the present application has excellent conversion ability before and after drug release, which can accelerate the drug release in tumor cells.

9. In Vitro Biological Safety Evaluation of the Prepared Peptides for the Drug-Load Micelles

Human umbilical vein endothelial cells (HUVECs) were cultured with DMEM medium containing 10% fetal bovine serum (FBS) and 1% penicillin/streptomycin (P/S) in the environment of 37° C., 5% CO₂ and saturated humidity. According to the growth condition of cells, they are subcultured after the cells covered the bottom of Petri dish, which usually take 2-3 days for subculture. Finally, HUVEC cells in logarithmic growth period were chosen for thiazolyl blue tetrazolium bromide (MTT) assay. At the same time, other two types of tumor cells, MCF-7 and PANC-1, were selected for comparison in the cytotoxicity of the prepared peptides for drug-loaded systems on tumor cells. The culture method of the above two types of tumor cells is the same as that of normal HUVEC cells, except that the DMEM medium is replaced by RPMI-1640 medium.

HUVEC cells were cultured in 96-well plates at cell density of 5×10³ cells/well for 4 hours in incubator. The lyophilized powder of the polypeptide was dissolved in the prepared DMEM medium to get the final sample concentration at 50, 100, 200, 400, 600 and 800 μg/mL respectively. Five parallel experiments were performed at each concentration. After the cells in 96-well plates were cultured for 24 hours, 200 μL of the culture medium containing 0.5 μg/mL MTT was added to each well. After the cells were cultured for 4 hours, all culture mediums containing MTT were removed, then 150 μL DMSO solution was added to each well. After the 96-well plate was agitated for a period, the OD value of each well was detected by a microplate reader at the wavelength of 570 nm. The cells with culture medium only were used as the negative control group, and cell-free culture medium was used as blank control.

Finally, the cell relative survival rates of different samples were calculated by the following formula:

Cell survival rate=(OD value of drug-treated group−OD value of cell-free culture medium)/(OD value of negative control groups−OD value of cell-free culture medium)×100%.

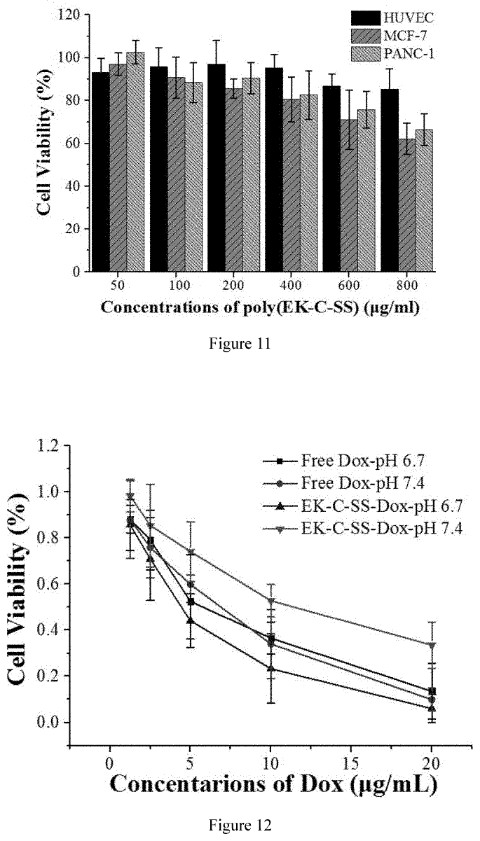

The typical cytotoxicity results of the p(EK-co-C) polypeptide with hydrazide groups via disulfide bonds are shown in FIG. 11, which indicates that the polypeptides did not show cytotoxicity under normal drug delivery concentration, and only inhibit cell activity at high concentration.

10. In Vitro Inhibition Efficacy of the Drug-Load Micelles Upon MCF-7 Cells

Breast cancer cells (MCF-7) were cultured in RPMI-1640 cell culture medium (containing 10% FBS and 1% P/S) at 5% CO₂ and 37° C. According to the growth condition of cells, they are subcultured after the cells covered the bottom of Petri dish, which usually take 2-3 days for subculture. Finally, MCF-7 cells in logarithmic growth period were chosen for MTT assay.

MCF-7 cells were cultured in 96-well plates at cell density of 5×10³ cells/well for 4 hours in incubator. The lyophilized powder of the nanodrug (EK-C-SS-Dox) was dissolved in RPMI-1640 medium with both pH of 6.7 (adjust pH with HCl) and 7.4 to get the final sample concentration at 1.25, 2.5, 5, 10 and 20 μg/mL (Dox equivalent concentration) respectively. RPMI-1640 cell culture mediums with free DOX at corresponding concentrations and pHs were also prepared as the positive control group. After the cells in the 96-well plate were cultured for 24 hours, the culture medium was discarded and replaced by the RPMI-1640 cell culture medium with EK-C-SS-Dox or free DOX (200 μL for each well). Five parallel experiments were performed at each concentration. After the cells were cultured for 4 hours, the medium containing drugs was discarded and replaced the RPMI-1640 cell culture medium for 20-hours incubation again. Then, 200 μL culture medium containing 0.5 μg/mL MTT was add to each well for 4-hour incubation, and was discarded and replaced by 150 μL DMSO. After the 96-well plate was agitated for a period, the OD value of each well was detected by a microplate reader at the wavelength of 570 nm. The cells with culture medium only were used as the negative control group, and cell-free culture medium was used as blank control.

The typical cytotoxicity results of the doxorubicin-conjugated nanodrug based on negative-charge-biased p(EK-co-C) polypeptide with hydrazide groups via disulfide bonds are shown in FIG. 12, which shows that comparing with free DOX, the drug loaded micelles have significantly improved in vitro inhibitory effect on breast cancer cells (MCF-7) when the physiological pH 7.4 is changed to the tumor microenvironmental pH 6.7

11. Cellular Internalization Mechanism of the Drug-Load Micelles by MCF-7 Cells

Refers to Example 5 (10) for the culture method of MCF-7 cells.

The internalization mechanism of drug loaded micelles was investigated by MCF-7 cells. The detailed procedure was as follows: MCF-7 cells were cultured in 6-well plates at cell density of 5×10⁵ cells/well for 20 hours in incubator. The RPMI-1640 cell culture mediums (pH 6.7, adjusted with HCl ) with cell inhibitors (30 μg/mL chlorpromazine hydrochloride, 15 μg/mL genistein, 133 μg/mL amiloride hydrochloride, 30 μg/mL and 7.5 mM methyl-β-Cyclodextrin, respectively) were prepared. Then additional lyophilized drug-loaded micelle was dissolved in the above cell culture mediums to get 10 μg/mL equivalent concentration of DOX. And the RPMI-1640 cell culture mediums in 6-well plates were discarded and replaced by 2 ml RPMI-1640 cell culture medium with additional inhibitors. After 30 minutes, the culture mediums were discarded and replaced by 2 ml RPMI-1640 cell culture medium with both additional inhibitors and the drug-loaded micelles. Then, the culture mediums were discarded again and the wells were washed by sterile PBS 2-3 times. After the extracellular matrix (ECM) were digested with trypsin, cells were collected by centrifuged to remove the supernatant and resuspended in 500 μL sterile PBS solutions. The flow cytometry (FCM) was used to record the internalization amount of the drug-loaded micelles. The group only treated by the drug-loaded micelles was used as the negative control group. Another group treated by the drug-loaded micelles without cell inhibitors was incubated in a refrigerator at 4° C. for 2 hours and 30 minutes. And then they were also analyzed by FCM. During FCM detection, 10000 cells were collected.

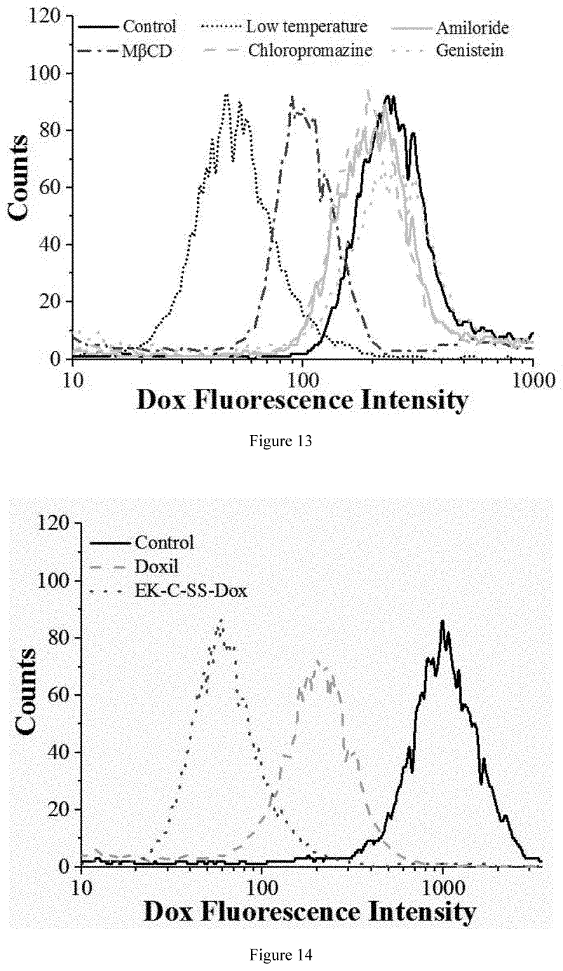

The typical cellular internalization results of the doxorubicin-conjugated nanodrug based on negative-charge-biased p(EK-co-C) polypeptide with hydrazide groups via disulfide bonds are shown in FIG. 13, indicating that the internalization of the nanodrugs is partially inhibited by methyl-β-cyclodextrin (MβCD) and completely inhibited at low temperature, and not obviously inhibited through other pathways. This suggests that the internalization of the nanodrugs relies on an energy-dependent mode through the hydrophobic interaction between the carrier and cell membrane.

12. Resistance to Immune Phagocytosis of the Drug-Loaded Micelles by RAW-264.7 Cells

RAW-264.7 cells were cultured in RPMI-1640 cell culture medium (containing 10% FBS and 1% P/S) at 5% CO₂ and 37° C. According to the growth condition of cells, they were subcultured after the cells covered the bottom of Petri dish. The cells were harvested by pipette blowing without trypsin treatment. Finally, RAW-264.7 cells in logarithmic growth period were chosen for resistance to immune phagocytosis assay.

RAW-264.7 cells were cultured in 6-well plates at cell density of 5×10⁵ cells/well for 20 hours in incubator. The RPMI-1640 cell culture medium with respectively lyophilized drug loaded micelle, Doxil and free Dox at 10 μg/mL equivalent concentration of DOX was prepared. And the RPMI-1640 cell culture mediums in 6-well plates were discarded and replaced by 2 ml RPMI-1640 cell culture medium with additional drugs for another 4-hour incubation. Then, the cell culture medium with additional drugs was discarded and washed by sterile PBS solutions twice. The cells were harvested by pipette blowing for 1-2 minutes and were collected by centrifuged to remove the supernatant and resuspended in 500 μL sterile PBS solutions. The flow cytometry (FCM) was used to record the internalization amount of the drug-loaded micelles. Free DOX group was used as the positive control group. During FCM detection, 10000 cells were collected.

The typical results of resistance to immune phagocytosis (RAW-264.7) of the doxorubicin-conjugated nanodrug based on negative-charge-biased p(EK-co-C) polypeptide with hydrazide groups via disulfide bonds are shown in FIG. 14, which shows that the resistance to immune phagocytosis of this carrier was significantly higher than that of commercial Doxil®.

13. In Vivo Clearance Assay of the Drug-Loaded Micelles in Mice

All experiments involving animals comply with the Animal Ethics Committee of Zhejiang University. ICR female mice weighing about 18-22 g were selected as models to study the plasma clearance of the drug-loaded micelles. The mice were randomly divided into 3 groups, with 3 mice in each group. The drug-loaded micelle lyophilized powder, commercially available Doxil, and free doxorubicin hydrochloride were dissolved in 1 xPBS solution, and each mouse was injected with 200 μL solutions at doxorubicin equivalent concentration of 10 mg/kg by tail vein injection. After administration of 2 min, 1 h, 2 h, 4 h, 6 h, 12 h and 24 h respectively, 20 μL of blood was taken from each mouse through orbital veins and put into a centrifuge tube containing heparin sodium, then 370 μL of acidified methanol solution was added to the centrifuge tube containing mouse blood. The centrifuge tube was put into a 37° C. constant temperature air bath shaker at 200 rpm for 24 h. Finally, the samples were centrifuged for 5 minutes at 4° C., 3500 rpm. The fluorescence value was measured with a fluorescence spectrophotometer at the excitation wavelength of 485 nm and the emission wavelength of 593 nm. Finally, the drug concentration in the blood at different time points was calculated according to the standard curve of free DOX in acidified methanol.

DOX standard curve was established with a fluorescence spectrophotometer as follows: DOX solutions dissolved in methanol solution containing 1% concentrated hydrochloric acid with concentrations of 0.1, 0.5, 1, 2.5, 5 and 10 μg/mL DOX were prepared respectively, and their the fluorescence values were measured under the condition of excitation wavelength of 485 nm and emission wavelength of 593 nm. Finally, the DOX fluorescence intensity vs concentration standard curve was drawn.

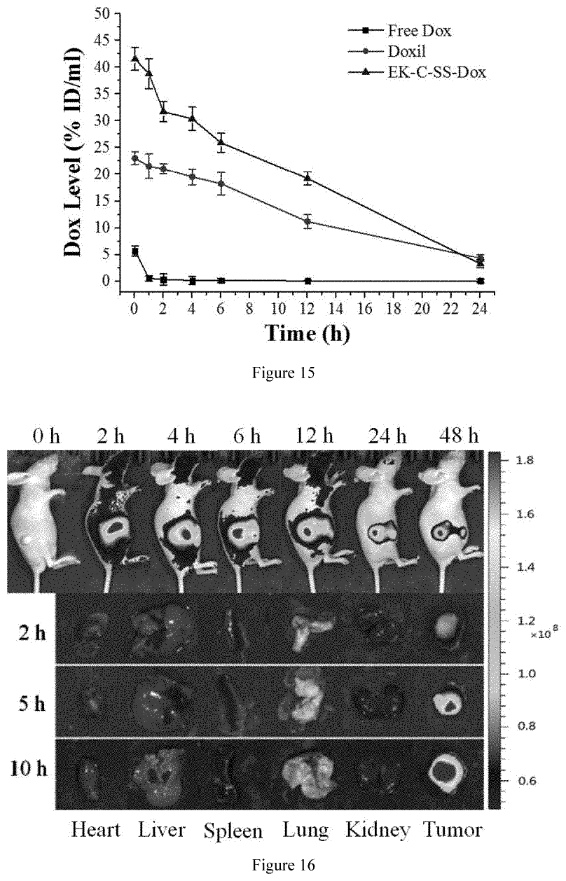

The typical in vivo clearance results of the doxorubicin-conjugated nanodrug based on negative-charge-biased p(EK-co-C) polypeptide with hydrazide groups via disulfide bonds are shown in FIG. 15, indicating that this carrier has longer blood circulation time than commercially available Doxil®, and significantly elevates the blood concentration of drugs.

14. In Vivo Biodistribution of the Drug-Loaded Micelles in Mice

Balb/c nude mice weighing 18-22 g were purchased from Shanghai Shrek animal Co., Ltd. Construction of tumor bearing nude mouse model: MCF-7 cells were cultured in RPMI-1640 cell culture medium (containing 10% FBS and 1% P/S) at 5% CO₂ and 37° C. When the cells proliferated to a certain amount, they were harvested through the digestion of ECM by trypsin at 37° C. for 2 minutes, which was terminated by additional FBS and centrifugation to remove the supernatant. 100 μL sterile PBS and 100 μL matrix glue mixed solution were added to resuspend the cells. 200 μL (about 5×10⁶ cells) cell solution was injected subcutaneously near the lower limbs of nude mice. When the tumor volume reached 50 mm³, the biodistribution experiment was carried out. The tumor volume was calculated according to the formula V=1/2*ab², where a was the long length of the tumor, and b was the short length of the tumor.

A certain amount of the lyophilized drug-loaded micelle, which was labeled with Cy5.5-NHS, was dissolved in sterile PBS solution. 200 μL of the above solution (DOX equivalent concentration was 8 mg/kg) were taken with a syringe, and injected into mice through tail vein. Mice were anesthetized with isoflurane gas at 2 h, 4 h, 6 h, 12 h, 24 h, 48 h post tail-vein-injection, and in vivo biodistribution of drugs was recorded by a near-infrared fluorescence imaging system (IVIS Lumina II).

The tumor-bearing mice were sacrificed through neck-breaking at 2 h, 5 h and 10 h post tail-vein-injection to obtain the tumor and organ tissues of the mice, which were recorded by a near-infrared fluorescence imaging system (IVIS Lumina II). And fluorescence quantitative correction was carried out with IVIS spectrum software to accurately calculate the concentration of drugs in various tissues and organs. The excitation wavelength and the emission wavelength of the instrument were set as 670 nm and 710 nm respectively, the exposure time was set as 4 seconds, and the aperture was set as a D mode.

The typical in vivo biodistribution results of the doxorubicin-conjugated nanodrug based on negative-charge-biased p(EK-co-C) polypeptide with hydrazide groups via disulfide bonds are shown in FIG. 16, which showed that the nanodrugs were mainly concentrated in tumors with a greatly decrease in the healthy organs, such as liver and kidney where show large amount accumulation of conventional nanodrugs. This showed excellent tumor targeting of this nanodrug in vivo.

15. In Vivo Tumor Inhibition Efficacy of the Drug-Loaded Micelles in Mice

Balb/c nude mice weighing 18-22 g were randomly divided into 3 groups with 6 mice in each group, of which 5 were used to monitor the weight change and survival rate of mice, and the other one was used for histopathological analysis. Then each group of mice was subcutaneously injected with 200 μL of PBS solution containing 5×10⁶ MCF-7 cells. When the tumor volume reached about 100 mm³, the nanodrug was injected via tail vein once every 4 days. The drug-loaded micelles with a dose of 8 mg/kg DOX equivalent concentration were served as the experimental group, the Doxil group with the same DOX equivalent concentration was served as the positive control group, and the PBS group was served as the negative control group. The weight changes of mice in each group were weighed and recorded at the next day of injection, and the long length (a) and short length (b) of tumors were measured with a vernier caliper to calculate and record the changes of tumor volume (V). After 18-day administration, one mouse from each group was sacrificed, and the major organs and tumors were harvested for histopathological analysis. The tumor volume (V) was calculated according to the formula as: V=1/2*ab², where a represented the long length of the tumor, and b represents the short length of the tumor.

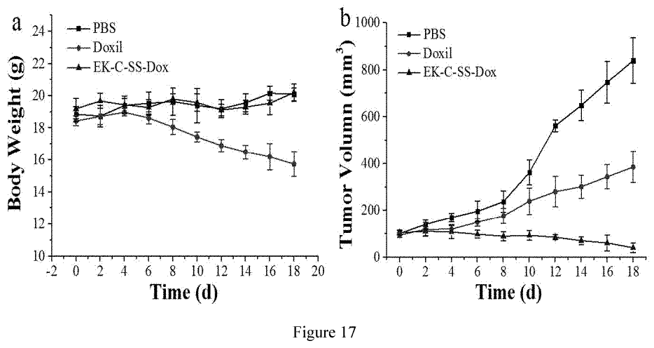

The typical in vivo antitumor efficacy results of the doxorubicin-conjugated nanodrug based on negative-charge-biased p(EK-co-C) polypeptide with hydrazide groups via disulfide bonds are shown in FIG. 17b (volume change of tumors borne on nude mice) and FIG. 17a (body weight change of the mice), indicating that the nanodrug had higher antitumor efficacy than Doxil®. The tumor volume treated with the nanodrugs was rapidly reduced with lower toxicity than Doxil®. The body weight change of nude mice was similar to that of PBS control group, demonstrating healthier condition of mice after treated by the nanodrug.

Any nanodrug prepared from example 1 to example 5 was characterized by the same methods as example 6, and their antitumor efficacy was similar, leading to the tumor growth significantly slower than that of the non-treatment group.

The above descriptions of the specific examples of the application in combination with the attached figures shall not be construed as limiting the scope of protection of the application. Obviously, the application is not limited by the above examples, and any modification or transformation based on the claims of the application shall be covered by the scope of protection of the application.

What is claimed is:

1. A zwitterionic polypeptide for negative-biased anti-tumor nanomedicines, wherein, the zwitterionic polypeptide has a structure represented by formula (I): 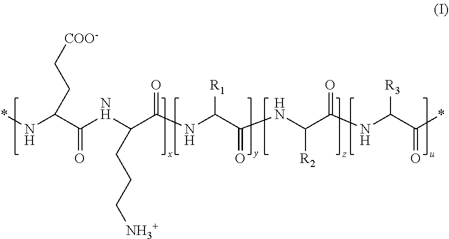
wherein, x, y, z and u each independently are positive integers, x≥2, y+z+u is greater than 0; R₁ is —CH₂SH and/or a derivative thereof; R₂ is —CH₂COO⁻ and/or an anionic derivative of —CH₂SH; R₃ is a group used to facilitate the degradation of peptides by enzymes.

2. The zwitterionic polypeptide of claim 1, wherein the zwitterionic polypeptide has a structure represented by any one of formulas II to VI: 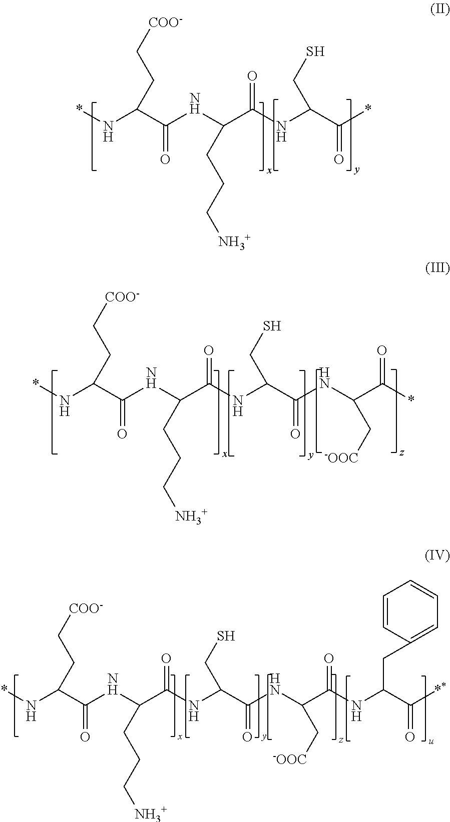 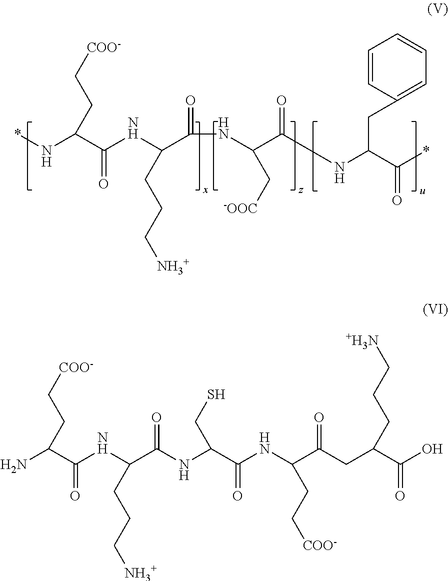
wherein, x, y, z and u each independently are positive integers, x≥2.

3. A derivative of the zwitterionic polypeptide of claim 2, wherein,
the derivative of the zwitterionic polypeptide represented by formula II has a structure represented by formula VII or VIII;
the derivative of the zwitterionic polypeptide represented by formula III has a structure represented by formula IX or X;
the derivative of the zwitterionic polypeptide represented by formula IV has a structure represented by formula XI or XII;
the derivative of the zwitterionic polypeptide represented by formula VI has a structure represented by formula XIII or XIV; 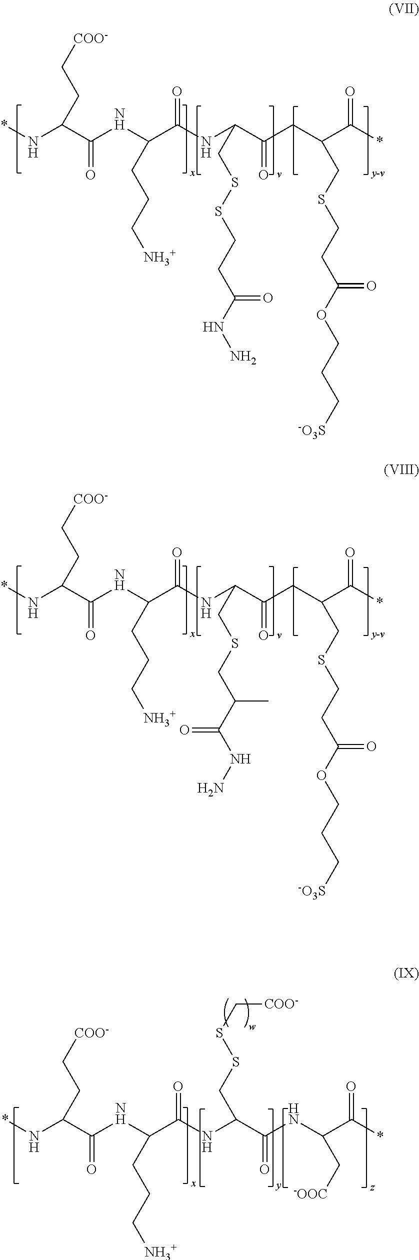 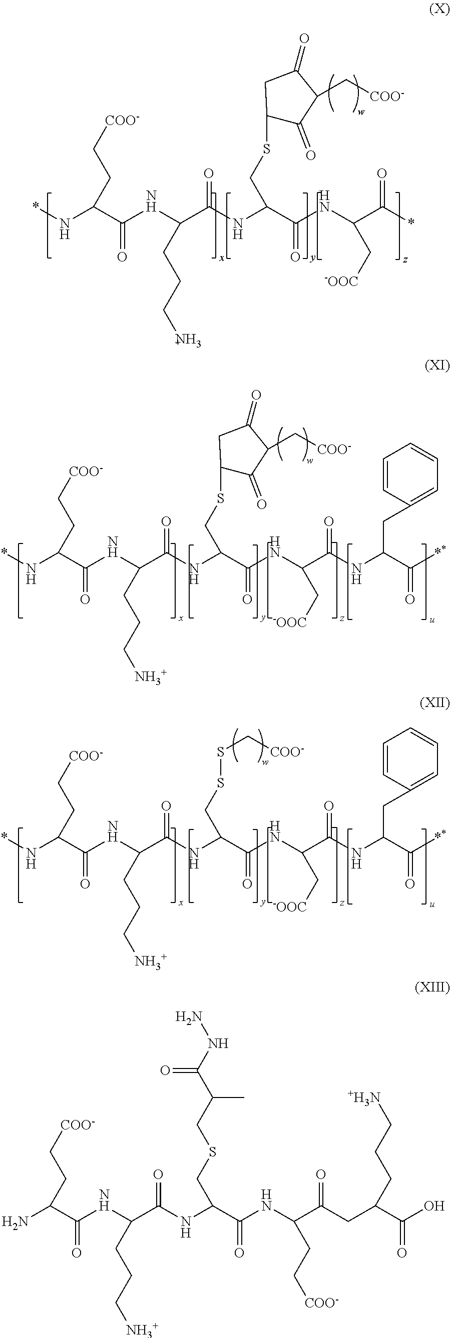 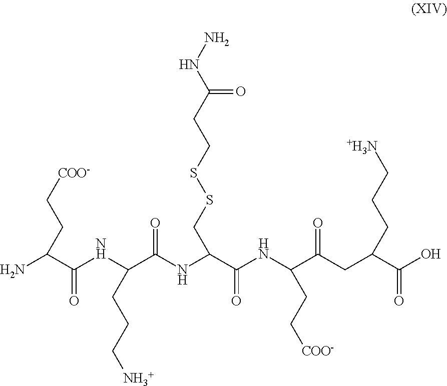
wherein, x, y, z and u each independently are positive integers, x≥2, and w>6.

4. The derivative of the zwitterionic polypeptide of claim 3, wherein a hydrazide group in the derivative represented by formula VII, VIII, XIII or XIV has a structure represented by formula XV after reaction with doxorubicin:

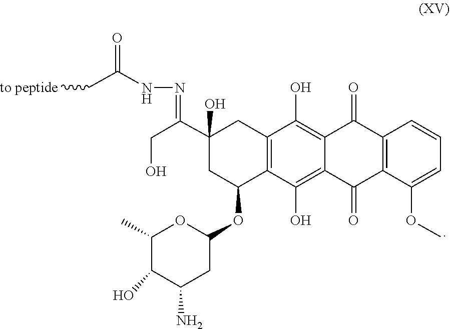

5. A method of preparing the zwitterionic polypeptides of claim 1, comprising:
allowing glutamyl-lysine dipeptide monomer containing side chain protecting groups to undergo mixed polycondensation reaction with any one or more of side-chain protected cysteine, side-chain protected aspartic acid, and phenylalanine, and deprotecting a mixed polycondensation product.

6. A nanodrug, obtaining from encapsulation of a hydrophobic drug by a hydrophobic side chain in the zwitterionic polypeptide represented by formula I, IV or V, or obtaining from encapsulation of the hydrophobic drug by a hydrophobic side chain in the derivative of the zwitterionic polypeptide represented by formula IX, X, XI or XII; 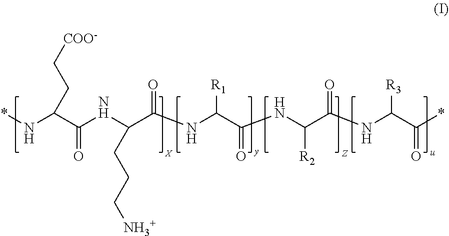
wherein, x, y, z and u each independently are integers, x≥2, y+z+u>0; R₁ is —CH₂SH and/or a derivative thereof; R₂ is —CH₂COO⁻ and/or an anionic derivative of —CH₂SH; R₃ is a group used to facilitate the degradation of peptides by enzymes; 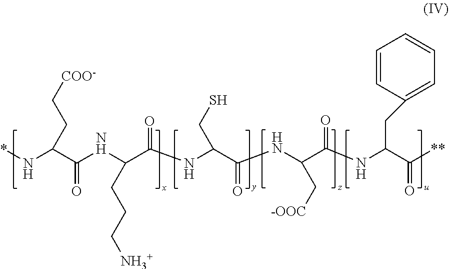
wherein, x, y, z and u each independently are positive integers, and x≥2; 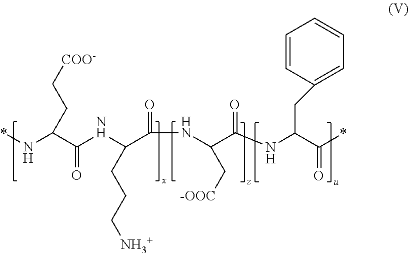
wherein, x, z and u each independently are positive integers, and x≥2; 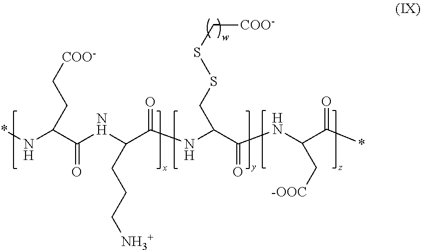
wherein, x, y, z and w each independently are positive integers, x≥2, and w>6; 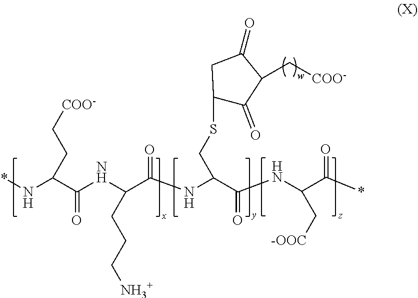
wherein, x, y, z and w each independently are positive integers, x≥2, and w>6; 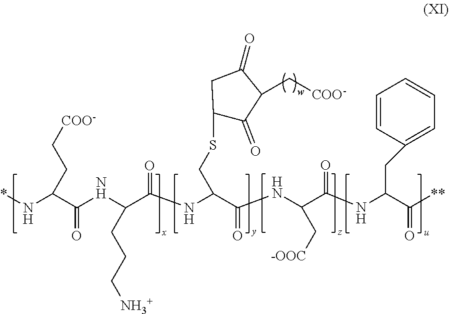
wherein, x, y, z, u and w each independently are positive integers, x≥2, and w>6; 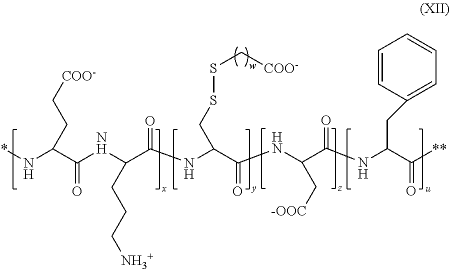
wherein, x, y, z, u and w each independently are positive integers, x≥2, and w>6.

7. The nanodrug of claim 6, wherein the nanodrug, obtained from the encapsulation of the hydrophobic drug by the hydrophobic side chain in the zwitterionic polypeptide represented by formula I, IV or IX, or in the derivative of the zwitterionic polypeptide represented by formula X, XI or XII, has a zwitterionic-polypeptide protective shell, which is formed by the cross-linking between cysteines in the corresponding zwitterionic polypeptide or the derivative of the zwitterionic polypeptide.

8. A nanodrug based on the derivative of the zwitterionic polypeptide of claim 4, wherein, the nanodrug is formed by the encapsulation of a hydrophobic drug by a hydrophobic side chain in the derivative of the zwitterionic polypeptide represented by formula XV, or formed by directly dispersing the derivative of the zwitterionic polypeptide represented by formula XV in a water solution.

9. The nanodrug of claim 6, wherein, a zeta potential of the nanodrug in a physiological solution of pH 7.4 is negative, and the zeta potential of the nanodrug in a 0.02 mol/L disodium phosphate-citric acid buffer solution of pH 6.7 is below 8 mV.

10. The nanodrug of claim 7, wherein, a zeta potential of the nanodrug in a physiological solution of pH 7.4 is negative, and the zeta potential of the nanodrug in a 0.02 mol/L disodium phosphate-citric acid buffer solution of pH 6.7 is below 8 mV.

11. The nanodrug of claim 8, wherein, a zeta potential of the nanodrug in a physiological solution of pH 7.4 is negative, and the zeta potential of the nanodrug in a 0.02 mol/L disodium phosphate-citric acid buffer solution of pH 6.7 is below 8 mV.

12. The nanodrug of claim 9, wherein, at least one negatively charged acid group in the nanodrug is more acidic than the carboxylic acid group of glutamate side chain, and/or a total amount of the negatively charged acid groups in the nanodrug is more than the amount of positively charged groups.

13. The nanodrug of claim 10, wherein, at least one negatively charged acid group in the nanodrug is more acidic than the carboxylic acid group of glutamate side chain, and/or a total amount of the negatively charged acid groups in the nanodrug is more than the amount of positively charged groups.

14. The nanodrug of claim 11, wherein, at least one negatively charged acid group in the nanodrug is more acidic than the carboxylic acid group of glutamate side chain, and/or a total amount of the negatively charged acid groups in the nanodrug is more than the amount of positively charged groups.
